(19) United States
(12) Patent Application Publication
(10) Pub. No.: US 2024/0260875 A1
(43) Pub. Date: Aug. 8, 2024

(54) SYSTEM AND METHOD FOR REMOTELY MONITORING MUSCLE AND JOINT FUNCTION

(71) Applicant: University of Vermont and State Agricultural College, Burlington, VT (US)

(72) Inventors: Reed GURCHIEK, Essex Junction, VT (US); Ryan MCGINNIS, Shelburne, VT (US)

(21) Appl. No.: 18/560,644

(22) PCT Filed: May 12, 2022
(86) PCT No.: PCT/US22/29076

§ 371 Date: Nov. 13, 2023

Related U.S. Application Data
(60) Provisional application No. 63/187,889, filed on May 12, 2021.

(51) Int. Cl. A61B 5/22; A61B 5/00; A61B 5/11; A61B 5/389

(52) U.S. Cl. CPC A61B 5/224; A61B 5/1107; A61B 5/389; A61B 5/4528; A61B 5/6802; A61B 5/7264; A61B 2562/0219; A61B 2562/06

(57) ABSTRACT

A system for determining dynamics of a joint of an individual includes a first muscle contraction sensor configured to measure an excitation of a first muscle adjacent to a joint; a first movement sensor configured to measure movement on a first side of the joint; a second muscle contraction sensor configured to measure an excitation of a second muscle located adjacent to the joint; and a second movement sensor configured to measure movement on a second side of the joint. A machine learning processor is trained to determine a full set of excitation values based on an excitation value from the first muscle contraction sensor and an excitation value from the second muscle contraction sensor. A processor is configured to determine a joint moment based on values from the first and second movement sensors and the set of excitation values from the machine learning processor.

### CROSS-REFERENCE TO RELATED APPLICATIONS

This application claims priority to U.S. Provisional Application No. 63/187,889, filed on May 12, 2021, now pending, the disclosure of which is incorporated herein by reference.

### STATEMENT REGARDING FEDERALLY SPONSORED RESEARCH

This invention was made with government support under contract no. NNX15AP86H awarded by the National Aeronautics and Space Administration and contract no. R21EB027852 awarded by the National Institutes of Health. The government has certain rights in the invention.

### FIELD OF THE DISCLOSURE

The present disclosure relates to joint dynamics, and in particular, techniques for instrumentation of a joint of an individual and techniques for characterizing the joint.

### BACKGROUND OF THE DISCLOSURE

Remote patient monitoring promises to revolutionize patient care in multiple clinical contexts. In orthopedics, continuous monitoring of human joint and muscle tissue loading in free-living conditions will enable novel insight concerning musculoskeletal disease etiology. These developments are necessary for comprehensive patient characterization, progression monitoring, and personalized therapy. This vision has motivated many recent advances in wearable sensor-based algorithm development that aim to perform biomechanical analyses traditionally restricted to confined laboratory spaces. However, these techniques have not translated to practical deployment for remote monitoring. Several barriers to translation have been identified including complex sensor arrays.

### BRIEF SUMMARY OF THE DISCLOSURE

Embodiments of the present disclosure overcome the problems present in the prior art. Systems and methods according to the present disclosure include a techniques for estimating individual muscle force, work, and power as well as joint moment using a minimal array of wearable sensors. These variables provide important clinical insight related to the onset and progression of musculoskeletal disease as well as for monitoring recovery in a patient's rehabilitation program. The hybrid technique combines both physics and probabilistic models in a complementary fashion. Specifically, the presently-disclosed technique utilizes probabilistic models of muscle synergy functions that enable the estimation of unmeasured muscle excitations computed from surface electromyograms (EMG) using only a subset of measured excitations (from other synergistic muscles). In some embodiments, only two to four surface electrodes are necessary and may be embedded into a frame (e.g., a knee brace) along with two inertial sensors; one on the upper arm of the frame (e.g., attached to the thigh segment) and one on the lower arm of the frame (e.g., attached to the shank segment). Data from these two inertial sensors are used to estimate segment kinematics and muscle-tendon unit lengths using physics-based techniques and a model of the musculoskeletal geometry. These estimates of muscle excitation and muscle-tendon unit length are used as inputs for electromyography (EMG)-driven simulation of muscle contraction. Muscle force, power, and work as well as net joint moment are outputs from the simulation. Other techniques that use only physics-based simulation require too many sensors that prohibit instrumentation. Alternatively, other techniques that use only machine learning do not characterize individual muscle contraction dynamics and are less generalizable than physics-based methods. The present disclosure combines these two techniques wherein machine learning is used where the physics are insufficiently informed and not well understood.

The minimal wearable sensor array and associated algorithms could be deployed in a number of potential configurations. For example, for surgeries where knee bracing is prescribed post-surgery (e.g., anterior cruciate ligament (ACL) reconstruction), the EMG and movement sensors (e.g., gyroscope, accelerometer) required to enable the novel algorithm could be embedded within the knee brace and communicate their data to an on-board processing unit which could, in turn, communicate directly with a cloud data storage system or a mobile phone for collecting and aggregating the data. For surgeries that do not require bracing post-operation (e.g., total knee arthroplasty), wearable sensors could be worn directly on the user's skin or integrated within a fabric or neoprene knee sleeve. These sensors could store data locally for offline processing or stream data to a mobile phone for on-board processing. In all cases, this disclosed system presents a practically deployable mechanism for monitoring joint loading (e.g., knee joint loading) in patients and communicating these data to healthcare providers to inform treatment. There are currently no feasible approaches for remotely tracking measures of muscle force, power, and work or net joint moment. Current knee braces either provide no mechanism of patient monitoring, serving only to stabilize the knee joint, or do not enable the estimation of clinically relevant biomechanical variables. Our exemplary knee brace and estimation algorithm advance instrumented knee brace technology by enabling comprehensive patient characterization of individual muscle function and joint loading. For the end-user, this technology provides the means for improving the rehabilitation process beyond simply stabilizing the joint. Wearable sensors have been shown effective in providing this type of feedback, but current systems are not integrated and require the individual placement of multiple sensors on different parts of the body. These complex sensor arrays increase patient burden which may discourage use. An instrumented brace overcomes this problem as the sensing technology is incorporated all-in-one into a single, easy-to-use bracing mechanism that is often prescribed by a clinician.

### DESCRIPTION OF THE DRAWINGS

For a fuller understanding of the nature and objects of the disclosure, reference should be made to the following detailed description taken in conjunction with the accompanying drawings, in which:

FIG. 1: Schematic overview of the proposed technique. Gyroscope and accelerometer data from thigh- and shank-worn IMUs (red sensors, upper left) drive the system kinematics (inertial motion capture) from which the required MTU lengths are computed. EMG data from a subset of instrumented muscles (black sensors, upper left) are mapped to the required full set of excitations through Gaussian Process models of the associated muscle synergy functions. The MTU lengths and excitation signals then drive the simulation of muscle contraction from which the biomechanical variables of interest are computed.

FIG. 2: Foot-ground contact model for estimating foot pitch angle (γ) in the IMC analysis. Ankle joint center position during stance was available as described in the main text. Planar translation was assumed and thus only the world frame {W} vertical and horizontal coordinates were needed. Ankle joint center height at mid-stance (middle) was set equal to that in the reference configuration (black dashed line) from which the positions of the initial stance rotation point (red dot, inferior to the heel) and terminal stance rotation point (green dot, inferior to the toe) were computed. Assuming rotation was about these points in the respective intervals, γ was computed as the angle between the rotation point-to-joint center vectors at each time instant during initial stance (red dashed arrow) or terminal stance (green dashed arrow) and the same in the reference configuration (solid red and green arrows at mid-stance).

FIG. 3 depicts a knee brace system according to an embodiment of the present disclosure.

FIG. 4 depicts a knee sleeve system according to another embodiment of the present disclosure.

FIG. 5 depicts the knee sleeve of FIG. 4 shown as it would be worn.

FIG. 6 depicts a knee brace system according to another embodiment of the present disclosure.

FIG. 7 is a visual overview of the an embodiment of the present estimation procedure. In this example, four muscle excitation time-series are available from measured sEMG data (input muscles: vᵢ) and are used to estimate the excitation time-series of six other muscles (output muscles: yᵢ). To estimate the muscle excitation at time t (green dashed line) for a given output muscle, a finite time interval [t₁ tₙ] (black dashed lines), called the input window (shaded red area), of each input muscle may be input to the corresponding synergy function (ƒᵢ).

FIG. 8. Relationship between window size (in seconds) and z-scores used to rank window structures for window relative output times t=tₙ (dashed grey line) and t=0.5(t₁+tₙ) (solid black line) using the best four-muscle input set (BF, PL, SOL, VL: leftmost plot), the best three-muscle input set (BF, PL, SOL: middle plot), and the best two-muscle input set (LG, SOL: rightmost plot).

FIG. 9. Graphical comparison of the estimated excitation time-series (red line) and the measured excitation time-series (black line) for data from the test set of a typical subject. The shaded area represents the mean±two standard deviations, i.e., \sqrt{var(yₘ*)} in eq. (14).

FIG. 10. Graphical comparison of muscle activation time-series from estimated (red lines) and measured (black lines) muscle excitation time-series per the analysis described in section 1.III.E. Shown here are the middle five seconds of each activation time-series determined using a second-order, linear activation model driven by corresponding excitations from the test set. Columns correspond to individual subjects and rows correspond to individual output muscles. Activation units have been normalized by the average activation throughout the full 15 seconds of test set data.

FIG. 11. Ensemble average net KFM. Positive (negative) values indicate a flexion (extension) moment. The shaded area is ±1 s.d. (ID, IMC-GP).

FIG. 12. Scatter plot of peak KEM from the proposed technique (IMC-GP) compared to the ground truth inverse dynamics (ID) analysis. The 27 datapoints represent three for each of the nine subjects.

FIG. 13. Scatter plots of cumulative muscle work (three-trial average) from the proposed technique (IMC-GP) compared to the reference EMG-driven technique (OMC-Full). Work is shown for the type of contraction in which each muscle did the most work: concentric (triangles) or eccentric (circles). Bold titles indicate muscles for which measured excitations were used to simulate contraction whereas the others were based on the GP synergy function models.

### DETAILED DESCRIPTION OF THE DISCLOSURE

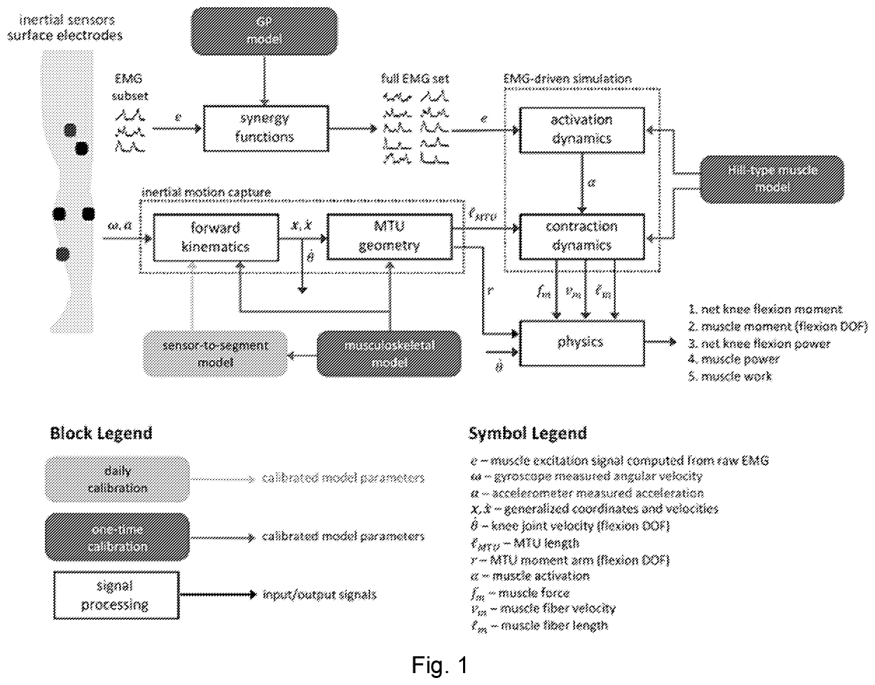

In the present disclosure, systems and methods are described to characterize joint and/or muscle dynamics. In some embodiments, Gaussian process (GP) synergy functions were used to estimate a complete set of muscle excitations using only a measured subset thus reducing the number of required electromyography (EMG) sensors. These excitations were used along with estimates of muscle-tendon unit (MTU) kinematics from inertial measurement unit (IMU) data in an EMG-driven simulation to characterize the joint and muscle dynamics (FIG. 1).

In a first aspect, the present disclosure may be embodied as a system for determining dynamics of a joint of an individual. In exemplary embodiments throughout the disclosure, reference is made to exemplary systems for determining dynamics of a knee joint of a human; however, such exemplary systems are intended to be non-limiting. Embodiments of the presently-disclosed system may be used for other joints of a human and/or non-human individual.

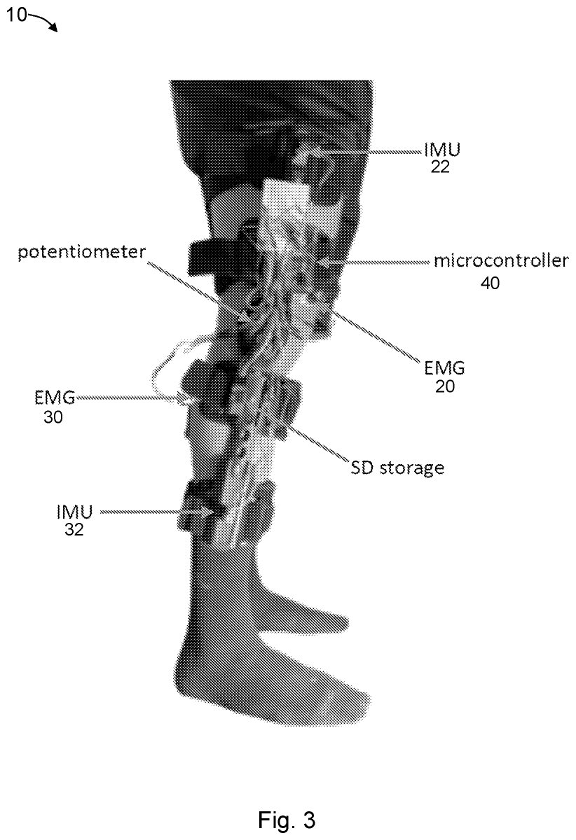

With reference to FIG. 3, a system 10 for determining dynamics of a joint of an individual includes a first muscle contraction sensor 20. The first muscle contraction sensor (or any muscle contraction sensor described herein) may be an electromyography (EMG) sensor. For convenience and clarity, reference is made herein (including in the attachment) to EMG sensors. However, unless expressly stated otherwise, any technology for characterizing muscle contractions (e.g., electromyography, mechanomyography, etc.) may be used and is within the scope of the present disclosure. The first muscle contraction sensor is configured to measure an excitation of a first muscle located adjacent to the joint to be measured (joint of interest). By adjacent muscle, the first muscle is on a segment (e.g., a muscle-tendon unit (MTU) segment) adjacent to the joint of interest. For example, where the knee joint is to be characterized, the first muscle contraction sensor may be located above the knee to measure an excitation of a muscle above the knee or the first muscle contraction sensor may be located below the knee to measure an excitation of a muscle located below the knee.

A first movement sensor 22 is located on a first side of the joint. The first side of the joint is a location proximate to the joint—e.g., without another joint between the first side location and the joint of interest. The first movement sensor is configured to measure movement on the first side of the joint. The first side may be the same side of the joint as the first muscle contraction sensor, or it may be an opposite side of the joint from the first muscle contraction sensor. In some embodiments, the first movement sensor is configured to measure movement in at least six degrees of freedom. The first movement sensor (or any movement sensor described herein) may be an inertial movement unit (IMU). For convenience and clarity, reference is made herein (including in the attachment) to IMUs. However, unless expressly stated otherwise, any technology for quantifying joint kinematics (thus, MTU dynamics) (e.g., electrogoniometers, string potentiometers with string routed over the knee cap, etc.) may be used and is within the scope of the present disclosure. In some embodiments, the first movement sensor includes at least one gyroscope and at least one accelerometer e.g., IMU includes at least one gyroscope and at least one accelerometer.

The system 10 includes a second muscle contraction sensor 30 (e.g., electromyography sensor, mechanomyography sensor, etc.), which is configured to measure an excitation of a second muscle. The second muscle contraction sensor is configured to measure a second muscle located adjacent to the joint to be measured (joint of interest). By adjacent muscle, the second muscle is on a segment (e.g., a muscle-tendon unit (MTU) segment) adjacent to the joint of interest. For example, where the knee joint is to be characterized, the second muscle contraction sensor may be located above the knee to measure a muscle above the knee or the second muscle contraction sensor may be located below the knee to measure a muscle located below the knee. The second muscle may be located on the same side of the joint as the first muscle or an opposite side of a joint form the first muscle.

A second movement sensor 32 (e.g., IMU) is located on a second side of the joint. The second side of the joint is a location proximate to the joint—e.g., without another joint between the second side location and the joint of interest—and across the joint from the first side. The second movement sensor is configured to measure movement on the second side of the joint. In some embodiments, the second movement sensor is configured to measure movement in at least six degrees of freedom. In some embodiments, the second movement sensor includes at least one gyroscope and at least one accelerometer.

Additional muscle contraction sensors and/or movement sensors may be utilized to measure muscles and/or movements of the joint. For example, a third muscle contraction sensor may be configured to measure an excitation of a third muscle located adjacent to the joint (e.g., on the same side of the joint as the first and/or second muscle contraction sensors or opposite side of the joint from the first and/or second muscle contraction sensors). In another example, a fourth muscle contraction sensor may be configured to measure an excitation of a fourth muscle located adjacent to the joint (e.g., on the same side of the joint as one or more of the first, second, and third muscle contraction sensors or an opposite side of the joint from one or more of the first, second, and third muscle contraction sensors).

The system 10 includes a processor 40 configured to determine a moment of the joint (joint moment). In the embodiment shown in FIG. 3, the processor 40 includes a machine learning processor. In some embodiments, the machine learning processor is separate from the processor. In some embodiments, the machine learning processor is a sub-processor of the processor. In some embodiments, the machine learning processor is embodied as programming of the processor. The machine learning processor is trained to determine a set of excitation values, wherein the set of excitation values includes an excitation value for each muscle of the joint (e.g., a “full set” or “complete set” of values). In other words, each excitation value of the set of excitation values corresponds to a different muscle of the joint such that each muscle of the joint has an excitation value. As described above, each muscle of the joint is each muscle which is relevant to movement of the joint of interest—e.g., adjacent to the joint of interest. The set of excitation values for each muscle of the joint is determined based on excitation values received from the first and second muscle contraction sensors. In embodiments with additional muscle contraction sensors, determining the set of excitation values may utilize excitation values from such additional muscle contraction sensors (e.g., third muscle contraction sensor, fourth muscle contraction sensor, etc.). In some embodiments, Gaussian process (GP) synergy functions (further described in the attachment) are used to estimate a complete set of muscle excitations using only a measured subset thus reducing the number of required muscle contraction sensors.

The machine learning processor may also utilize motion values from the first and/or second movement sensors to determine the set of excitation values. The machine learning processor may be trained by the individual performing a set of training motions. For example, the individual may complete a pre-determined set of training motions. In some embodiments, the machine learning processor has been trained using movement data from a plurality of individuals. In this way the subject individual may not need to perform a pre-determined set of training motions or the individual may need to perform a reduced number of pre-determined training motions.

The processor may calculate a set of MTU lengths and moment arm values from the IMU values. The processor may calculate a set of muscle forces for each muscle of the joint of interest. For example, the processor may determine the set of muscle forces based on muscle activation dynamics (e.g., from the set of excitation values determined by the machine learning processor) and known or calculated MTU lengths for the relevant muscles of the joint of interest. The joint moment may be determined based on the calculated set of muscle forces and moment arm values.

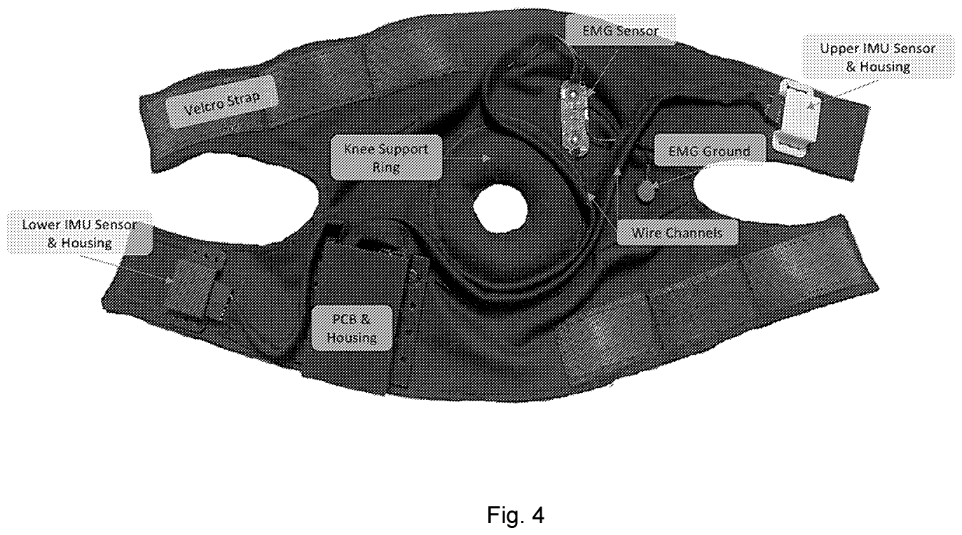

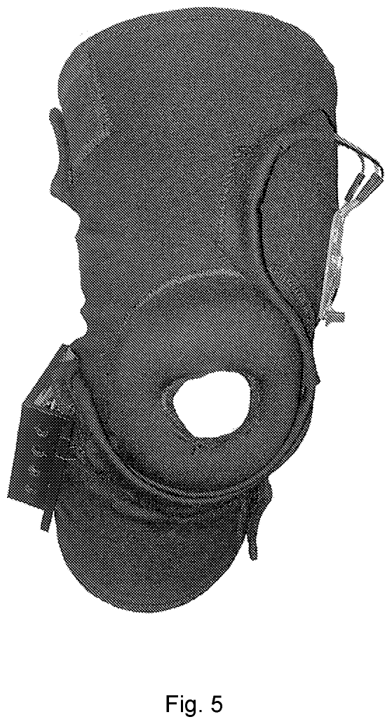

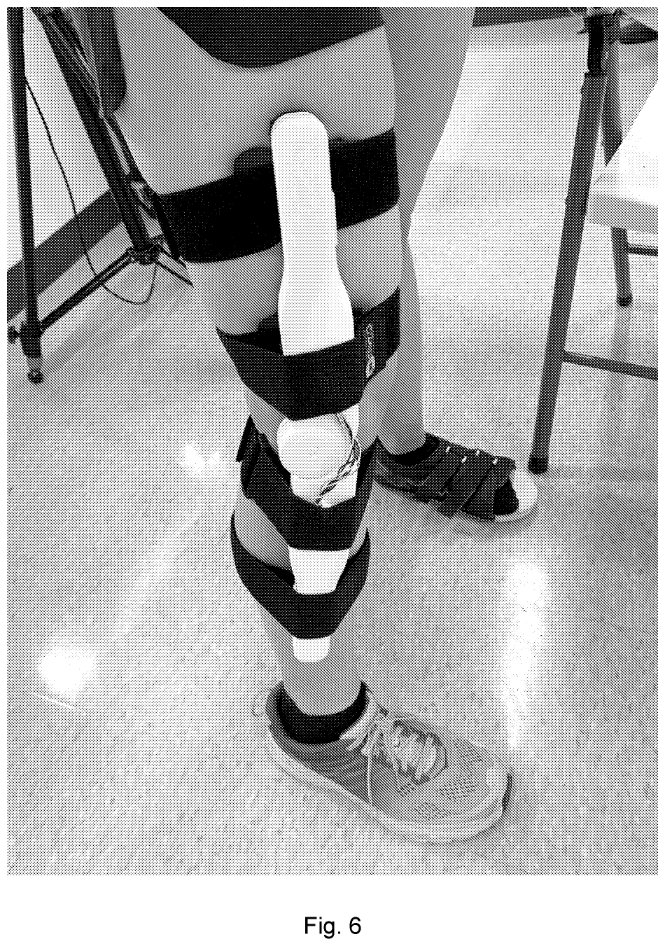

FIG. 3 depicts wherein a first EMG sensor, a second EMG sensor, a first IMU and a second IMU are each attached to a knee brace. Other embodiments of the system may include components attached to the individual using adhesives, straps, sleeves, bandages, wraps, etc. or combinations of such attachments. For example, the embodiment depicted in FIGS. 4 and 5 show the system embodied as a knee sleeve. FIG. 6 shows another embodiment of a system as a knee brace. In some embodiments, the processor (which may include the machine learning processor) is a remote processor. In some embodiments, the machine learning processor is a remote processor. For example, the processor and/or machine learning processor may be in a smartphone, a computer, a cloud device, etc. For example, where the processor and/or machine learning processor is in a smartphone, the processor and/or machine learning processor may be a part of the existing smartphone processor or may be separate from the existing smartphone processor.

In another aspect, a method for determining joint dynamics includes receiving data from muscle contraction sensors (e.g., EMG sensors, etc.) and movement sensors (e.g., IMUs, etc.) in a system as described above. For example, data may be received from a first EMG sensor configured to measure an excitation of a first muscle adjacent to a joint of interest, a second EMG sensor configured to measure an excitation of a second muscle located adjacent to the joint, a first IMU configured to measure movement on a first side of the joint, and a second IMU configured to measure movement on a second side of the joint opposite the first side. The method includes determining, using a machine-learning unit of a processor, a set of excitation values based on excitation values from the first and second EMG sensors, wherein the set of excitation values includes an excitation value for each muscle of the joint. The method may further include determining, using the processor, a set of muscle-tendon unit (MTU) lengths and moment arm values from the IMU values. The method may further include determining, using the processor, a set of muscle forces based on muscle activation dynamics (from the set of excitation values determined by the machine learning processor) and the MTU lengths.

In another aspect, the present disclosure may be embodied as a non-transitory computer-readable medium having stored thereon a computer program for instructing a computer to perform any of the methods disclosed herein. For example, a non-transitory computer-readable medium may include a computer program to receive data from a first muscle contraction sensor configured to measure an excitation of a first muscle adjacent to a joint, a second muscle contraction sensor configured to measure an excitation of a second muscle adjacent to the joint, a first movement sensor configured to measure movement on a first side of the joint, and a second movement sensor configured to measure movement on a second side of the joint opposite the first side.

The processor may be in communication with and/or include a memory. The memory can be, for example, a random-access memory (RAM) (e.g., a dynamic RAM, a static RAM), a flash memory, a removable memory, and/or so forth. In some instances, instructions associated with performing the operations described herein (e.g., calculating muscle excitation values, MTU lengths, etc.) can be stored within the memory and/or a storage medium (which, in some embodiments, includes a database in which the instructions are stored) and the instructions are executed at the processor.

In some instances, the processor includes one or more modules and/or components including, for example, machine-learning modules and/or components. Each module/component executed by the processor can be any combination of hardware-based module/component (e.g., a field-programmable gate array (FPGA), an application specific integrated circuit (ASIC), a digital signal processor (DSP)), software-based module (e.g., a module of computer code stored in the memory and/or in the database, and/or executed at the processor), and/or a combination of hardware- and software-based modules. Each module/component executed by the processor is capable of performing one or more specific functions/operations as described herein. In some instances, the modules/components included and executed in the processor can be, for example, a process, application, virtual machine, and/or some other hardware or software module/component. The processor can be any suitable processor (or more than one processor) configured to run and/or execute those modules/components. The processor can be any suitable processing device configured to run and/or execute a set of instructions or code. For example, the processor can be a general purpose processor, a central processing unit (CPU), an accelerated processing unit (APU), a field-programmable gate array (FPGA), an application specific integrated circuit (ASIC), a digital signal processor (DSP), and/or the like.

Some instances described herein relate to a computer storage product with a non-transitory computer-readable medium (which can also can be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also can be referred to as code) may be those designed and constructed for the specific purpose or purposes. Examples of non-transitory computer-readable media include, but are not limited to: magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; carrier wave signal processing modules; and hardware devices that are specially configured to store and execute program code, such as Application-Specific Integrated Circuits (ASICs), Programmable Logic Devices (PLDs), Read-Only Memory (ROM) and Random-Access Memory (RAM) devices. Other instances described herein relate to a computer program product, which can include, for example, the instructions and/or computer code discussed herein.

Examples of computer code include, but are not limited to, micro-code or micro-instructions, machine instructions, such as produced by a compiler, code used to produce a web service, and files containing higher-level instructions that are executed by a computer using an interpreter. For example, instances may be implemented using Java, C++, NET, or other programming languages (e.g., object-oriented programming languages) and development tools. Additional examples of computer code include, but are not limited to, control signals, encrypted code, and compressed code.

### Further Discussion

The following two sections provide additional discussion and describe experimental embodiments of the present disclosure. The described embodiments are non-limiting, and various examples are discussed each of which are included within the scope of the present disclosure.

### Section 1

### 1.I. Introduction

Recent developments in remote gait analysis point toward the need to incorporate free-living observation of joint mechanics into comprehensive patient evaluations. These measurements could capture patient-specific responses to prescribed interventions with an observation frequency not possible using laboratory-based approaches. Moreover, these measurements of clinically relevant biomechanics could be used to inform patient-specific modifications to rehabilitation programs and to evaluate their efficacy across a broad range of clinical populations.

Analysis of muscle excitations during gait are integral to delivering on this vision. In this disclosure, muscle excitation (a.k.a. sEMG amplitude) refers to the time-varying excited state of muscle and is related to motor unit recruitment and firing rate. Muscle excitation is distinguished from muscle activation which is defined functionally for scaling active muscle force in sEMG-driven muscle force estimation and has been related to the calcium dynamics promoting muscle force production. Muscle excitations alone provide clinical insight into motor control. To this end, they may be used to quantify control complexity in patients with neurological disorders via synergy analysis and, in a remote gait analysis application, for monitoring rehabilitation progress following knee surgery. Further, excitations drive muscle activation dynamics which may be used to estimate muscle forces. Remote estimation of muscle forces provides an avenue for advancing the current state of remote monitoring techniques (mostly limited to spatiotemporal variables) to incorporate additional clinically relevant biomechanical variables. For example, muscle work could be utilized in an adaptive rehabilitation context for its relation to work-induced muscle hypertrophy. Further, muscle forces are necessary to estimate muscle contributions to joint contact force and net joint moments using force plate-free techniques (a requirement for remote monitoring) both of which provide invaluable insight in many orthopedic conditions.

Wearable sEMG sensors provide the hardware solution to realize the aforementioned remote analyses, but one of the primary limitations to practical deployment is the number of sensors necessary. For joint contact force and net joint moment estimates, a sensor would be required on every muscle. Such a complex wearable system greatly impedes users' daily life; an increased burden that may discourage use. Non-sEMG data have been used to estimate muscle forces and/or excitations (with or without sEMG data) using both regression and optimization-based solutions to the muscle redundancy problem following inverse-dynamics. However, wearable sensor solutions in this context also require many sensors limiting use in remote gait analysis.

Regression techniques have been proposed as a means to reduce the number of necessary wearable sensors for estimating biomechanical time-series. These have proven successful for estimating joint moments, which as discussed earlier, serve as the starting point for optimization-based muscle force solutions. However, a hybrid approach may enable a more generalized solution wherein machine learning is used to augment estimation where the physics are least well understood. For example, consider that a model using only sEMG inputs trained to estimate joint angle during open-chain tasks must learn (1) the mapping from a subset of measured excitations to a complete set, (2) muscle activation dynamics, (3) muscle contraction dynamics, (4) rigid body dynamics, and (5) forward integration of the kinematic equations. In this example, steps (2)-(5) and their associated physics have been well studied whereas the mapping described in step (1) is least well understood. Thus, the presently-disclosed hybrid solution would be to approximate the behavior of step (1) using machine learning wherein a complete set of muscle excitations would be informed by a measured subset. This complete set of excitations could then drive the dynamics of steps (2)-(5) using sEMG-driven techniques. The success of regression techniques motivates the existence of the mapping in step (1) (which we refer to as synergy functions) and is further supported by recent results suggesting a subset of muscles may carry the information necessary for reconstructing unmeasured excitations.

In some embodiments of this disclosure, we model synergy functions as a Gaussian process (GP) and develop an approximation to their behavior that allows estimation of unmeasured excitations from a measured subset. Gaussian process regression is suitable in this context as it has been shown advantageous for small datasets and because it models the variance of the estimate which may prove beneficial in sensor fusion frameworks. Further, a GP model permits a connection to probabilistic theories of motor control. The novel developments in this study include the Gaussian process model of muscle synergy functions and the estimation of unmeasured muscle excitations using only a subset of sEMG data.

### 1.II. Gaussian Process Model of Synergy Functions

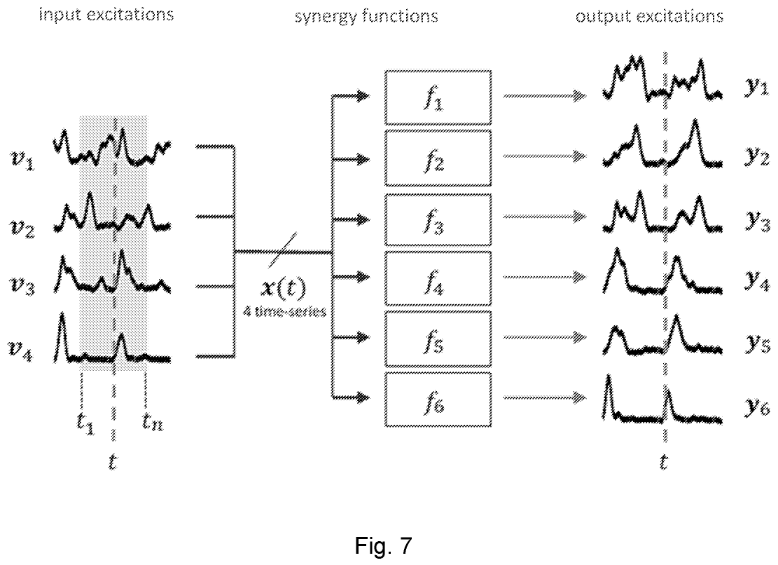

The main assumption made in this development is the existence of a function, ƒₘ: [symbol] ^{d}→ [symbol] , which maps the excitation yₘ(t)∈ [symbol] of muscle m (called the output muscle) at time t from an input vector x(t)∈ [symbol] ^{d} (FIG. 7). Specifically, x(t) is composed of excitations from a subset of p muscles (called the input muscle set) during a finite time interval, [t₁ tₙ], such that t₁≤t≤tₙ (called the input window). The d-dimensional input x(t) is partitioned as per,

$$x ( t ) = [ {ν}_{1} ( t ) {ν}_{2} ( t ) ⋯ {ν}_{p} ( t ) ] \qquad ( 1 )$$

where vᵢ(t) is n-dimensional,

$${ν}_{i ( t )} = [ {v}_{i , 1} {v}_{i , 2} ⋯ {v}_{i , n} ] \qquad ( 2 )$$

and v_{i,j} is the j^{th} sample of the excitation time-series of the i^{th} input muscle such that v_{i,1}=vᵢ(t₁) and v_{i,n}=vᵢ(tₙ). Thus, the dimension of the input, d=pn, is dependent on the number of input muscles, the size of the input window (tₙ−t₁), and the sEMG sampling frequency. The idea employed here is that the subset of input muscles and the output muscle are controlled in a coordinated fashion (i.e., synergistically) to achieve some sub-task during the time interval [t₁ tₙ] (e.g., propulsion of the center of mass during walking) and that this relationship can be inferred just from the excitations of the input muscles. Due to the nature of this mapping, we refer to ƒₘ as a synergy function.

Our approach is to approximate ƒₘ using Gaussian process regression [31]. To this end, we model the synergy function ƒₘ as a GP indexed on the input muscle excitations. This implies [32] that the scalar ƒₘ(x) is a random variable with Gaussian distribution as per ƒₘ(x)˜ [symbol] (μₘ(X),σₘ²(x)) and is jointly Gaussian with ƒₘ(x′) (a synergy function output associated with an arbitrary input vector x′) with covariance given by,

$$cov ( {f}_{m} ( x ) , {f}_{m} ( {x}^{′} ) ) = {k}_{m} ( x , {x}^{′} ) \qquad ( 3 )$$

where kₘ(x,x′) is a muscle-specific covariance function defining the covariance between ƒₘ(X) and ƒₘ(x′). Thus, for consistency, the variance of ƒₘ(x), denoted as σₘ²(x), is kₘ(x,x) and the mean is defined by a muscle-specific mean function μₘ(x).

To learn the behavior of the synergy function ƒₘ, we observe the synergistic relationship between the input muscle excitations and those of the output muscle given a set of N measured input-output pairs, (y_{m,i},xᵢ), called the training set. It is assumed that the measured output muscle excitations y_{m,i} in the training set are additively corrupted with muscle-specific, independent and identically distributed, zero-mean Gaussian noise, w_{m,i}˜ [symbol] (0,ϵₘ², so that,

$${y}_{m , i} = {f}_{m} ( {x}_{i} ) + {w}_{m , i} \qquad ( 4 )$$

The N measured output muscle excitations in the training set are used to form a column vector yₘ (a random vector),

$${y}_{m} = {[ {y}_{m , 1} {y}_{m , 2} ⋯ {y}_{m , N} ]}^{T} \qquad ( 5 )$$

which is characterized by a joint multivariate Gaussian distribution, yₘ˜ [symbol] (μₘ,Σₘ), expressed in terms of its mean vector μₘ and N×N covariance matrix Σₘ. Since w_{m,i} is zero-mean, the mean vector μₘ is (element-wise expectation in (5)),

$${μ}_{m} = {[ {μ}_{m} ( {x}_{1} ) {μ}_{m} ( {x}_{2} ) ⋯ {μ}_{m} ( {x}_{N} ) ]}^{T} \qquad ( 6 )$$

and due to the independence assumption, the element in row r and column c of Σₘ is,

$${[ {\sum }_{m} ]}_{r , c} = {k}_{m} ( {x}_{r} , {x}_{c} ) + {{ϵ}_{m}^{2} [ I ]}_{r , c} \qquad ( 7 )$$

where [I]_{r,c} is the element in row r and column c of the N×N identity matrix I.

Given an unseen input, x, (not in the training set), the corresponding synergy function value, ƒₘ*=ƒₘ(x_{*}), must also be jointly Gaussian with yₘ (per the GP model) and thus,

$$[ \begin{matrix}{y}_{m} \\ {f}_{m}^{*}\end{matrix} ] \sim 𝒩 ( [ \begin{matrix}{μ}_{m} \\ {μ}_{m} ( {x}_{*} )\end{matrix} ] , [ \begin{matrix}{Σ}_{m} & {k}_{m} ( X , {x}_{*} ) \\ {k}_{m}^{T} ( X , {x}_{*} ) & {k}_{m} ( X , {x}_{*} )\end{matrix} ] ) \qquad ( 8 )$$

where we have used kₘ^{T}(X,x_{*}) to denote the N-element row vector of covariances between ƒₘ* and each muscle excitation in the training set yₘ,

$${k}_{m}^{T} ( X , {x}_{*} ) = [ {k}_{m} ( {x}_{1} , {x}_{*} ) ⋯ {k}_{m} ( {x}_{N} , {x}_{*} ) ] . \qquad ( 9 )$$

and the symbol X is used to denote all input vectors in the training set. Finally, the conditional distribution of the synergy function value ƒₘ* is given by conditioning the joint Gaussian prior in (8) on the observed training data, yₘ. This conditional distribution is Gaussian with mean,

$${\overline{f}}_{m}^{*} = {μ}_{m} ( {x}_{*} ) + {k}_{m}^{T} ( X , {x}_{*} ) {\sum }_{m}^{- 1} ( {y}_{m} - {μ}_{m} ) \qquad ( 10 )$$

and variance,

$${( {σ}_{m}^{*} )}^{2} = {k}_{m} ( {x}_{*} , {x}_{*} ) - {k}_{m}^{T} ( X , {x}_{*} ) {\sum }_{m}^{- 1} {k}_{m} ( X , {x}_{*} ) . \qquad ( 11 )$$

Therefore, in the proposed estimation procedure, given a subset of (unseen) measured muscle excitations organized as a model input, x_{*}, our estimate of yₘ* is ƒ̅ₘ* in (10) and can be written as,

$${y}_{m}^{*} = {μ}_{m} ( {x}_{*} ) + {β}_{m , 1} {k}_{m} ( {x}_{1} , {x}_{*} ) + … + {β}_{m , N} {k}_{m} ( {x}_{N} , {x}_{*} ) \qquad ( 12 )$$

where the coefficient β_{m,i} (specific to output muscle m) is the i^{th} element of the column vector βₘ given by,

$${β}_{m} = {\sum }_{m}^{- 1} ( {y}_{m} - {μ}_{m} ) \qquad ( 13 )$$

and it is clear that the output yₘ* is a scalar. The variance of the estimate is the sum of the variance, (σₘ*)², of the conditional distribution of ƒₘ* in (11) and the noise variance ϵₘ² in (4),

$$var ( {y}_{m}^{*} ) = {( {σ}_{m}^{*} )}^{2} + {ϵ}_{m}^{2} . \qquad ( 14 )$$

When deploying this method in practice, the β_{m,i} are already known as they depend on training data only and as seen in (13) require inversion of the covariance matrix Σₘ. This inversion can be thought of as training the model and is required in (11) to describe the uncertainty of the estimate, yₘ*, in (14). Further, the mean function and covariance function may depend on one or more hyperparameters (including the noise variance Σₘ²) which are optimized by minimization of the negative log marginal likelihood over the training data using conjugate gradient descent. Hyperparameters for the models used in this work are introduced when the mean and covariance functions are specified. All aspects of training the GP models were done using the GPML toolbox in MATLAB R2019b. A custom package was developed that streamlines the development of different model structures (e.g., input muscle sets, input window size) for training, testing, and evaluation.

### 1.III. Experimental Validation

Sixteen unimpaired subjects walked for one-minute at a self-selected normal walking speed on a level treadmill (h/p/cosmos quasar) while sEMG data (BioStamp nPoint, MC10, Inc., sampling frequency: 1000 Hz) were continuously recorded from 10 muscles of the right leg: tibialis anterior (TA), peroneus longus (PL), lateral gastrocnemius (LG), medial gastrocnemius (MG), soleus (SOL), vastus medialis (VM), rectus femoris (RF), vastus lateralis (VL), biceps femoris (BF), and semitendinosus (ST). Electrode placement was according to SENIAM recommendations. Muscle excitations were computed from raw sEMG data using methods common for estimating muscle force. Specifically, sEMG data were digitally high-pass filtered at 30 Hz, rectified, low-pass filtered at 6 Hz, and normalized by the maximum value across the walking trial and several muscle-specific maximum voluntary contraction trials (MVC). All muscle excitation time-series were resampled to 250 Hz to explore application of the present approach for sampling frequencies used in remote monitoring. Following a visual sEMG data quality check, all data for seven subjects were removed as there was no clear signal during walking for at least one muscle. Thus, all data used in this study were from the other nine subjects (five males, height: 1.8±0.1 m, mass: 72.3±12.4 kg, age: 21±1 years). The average walking speed across all subjects was 0.84±0.13 m/s and the average stride time was 1.31±0.22 s.

The proposed GP model for estimating muscle excitations is modifiable in many ways. In this study, we explore the effects of different model characteristics at three levels: (1) input muscle sets, (2) structure of the input window, and (3) stationarity of the covariance function. All models explored were subject-specific models; i.e., all data used to train, and test a given model were from the same subject. Models were trained on 15 seconds of data from the first half of the one-minute walking trial. Models were evaluated using 15 seconds of data from the second half of the trial, and thus these data were not ‘seen’ by the model during the training process. A constant mean function was assumed for all GP models dependent on a single hyperparameter cₘ,

$${μ}_{m} ( x ) = {c}_{m} . \qquad ( 15 )$$

### A. Input Muscle Sets

The impact of input muscle set on estimation performance was explored using an exhaustive approach. We trained models using every possible combination of four, three, and two input muscles (out of the 10 we instrumented) yielding 210, 120, and 45 combinations, respectively. For each input set, GP models were trained to estimate the excitations of each of the muscles not in the input set. For this analysis, we used a scaled, squared exponential covariance function with isotropic length scale,

$${k}_{m} ( x , {x}^{′} ) = {θ}_{1} exp ( - \frac{{r}^{T} r}{2 {θ}_{2}^{2}} ) \qquad ( 16 )$$

where r=x−x′ and the hyperparameters are the scalar θ₁ and the characteristic length scale θ₂. The window size (tₙ−t₁) was set to 1.0 s and the window relative output time (tₙ−t) was set to 0.5 s. The maximum number of function evaluations for hyperparameter optimization was set to 50.

### B. Model Selection

Only one input muscle set for each of the four-, three-, and two-muscle input sets was used to further examine the effect of window structure (section 1.III.C.) and the stationarity of the covariance function (section 1III.D.). Using an exhaustive search approach, one would normally score the performance of each input set according to some performance metric and choose the one with the best score. However, several different metrics are common for evaluating estimation of biomechanical time-series including Pearson's correlation coefficient (r), percentage of variance accounted for (VAF), root mean square error (RMSE), and mean absolute error (MAE). In an attempt to select the best input set with consideration of each of these metrics we utilized a z-score averaging method. To this end, each metric was computed for each muscle-specific synergy function corresponding to each muscle in the output set for each investigated input set. These were evaluated by comparing the estimated excitations with the true measured excitations in the test set. Performance metrics for a given input set were first averaged across all output muscles and then across all subjects yielding four metrics (r, VAF, RMSE, MAE) for each input muscle set. Four additional metrics per input muscle set were computed wherein the same procedure was repeated except that muscles were weighted in the averaging across muscles according to their relative physiological cross-sectional area (PCSA). This was done to bias the selection of muscles with the greatest capacity to produce muscle force (on average). In practice, the excitations of the long head of the BF and of the ST are often assumed equivalent to that of the short head of the BF and the semimembranosus, respectively. Therefore, for the PCSA-weighted average, the BF weighting was based on the sum of the PCSA of the long and short heads of the BF and the ST weighting was based on the sum of the PCSA of the ST and the semimembranosus. Thus, eight performance metrics were available for each input muscle set. The values for each metric were converted to z-scores (demeaned and normalized by the standard deviation across all models). RMSE and MAE z-scores were negated so that larger values indicated better performance for all metrics. Finally, the average of these z-scores served as the single performance metric by which the models were ranked. This was done separately for the four-, three-, and two-muscle input sets under consideration.

### C. Input Window Structures

Window structure was modified according to the input window size (tₙ−t₁) and the window relative output time (tₙ−t). Fourteen different input window structures were explored according to every combination of seven input window sizes (1.75, 1.50, 1.25, 1.00, 0.75, 0.50, and 0.25 seconds) and two window relative output times: 0.0 s such that t=tₙ and half the window size such that t=0.5(t₁+tₙ). Each window structure was explored separately for the best four-, three-, and two-muscle input sets determined from the analysis described previously. The same mean function, covariance function, and hyperparameter optimization settings were used in this analysis. The best input window structure was chosen using the z-score averaging method in section 1.IIIB.

### D. Covariance Function Stationarity

In this analysis, we consider the stationarity of the chosen covariance function for the present estimation problem. The squared exponential covariance function in (16) is an example of a stationary covariance function (i.e., it is a function only of r=x−x′ which is unchanged given a translation of the origin). As an example non-stationary covariance function, we use the neural network covariance function,

$${k}_{m} ( x , {x}^{′} ) = {θ}_{1}^{2} arcsin ( \frac{{\tilde{x}}^{T} {\tilde{x}}^{′}}{\sqrt{( {θ}_{2}^{2} + {\tilde{x}}^{T} \tilde{x} ) ( {θ}_{2}^{2} + {\tilde{x}}^{′T} {\tilde{x}}^{′} )}} ) \qquad ( 17 )$$

where \tilde{x}^{T}=[x^{T} 1] and θᵢ are hyperparameters. It has been shown that (17) is the covariance function for a neural network with an error function activation in the hidden layer as the number of hidden units tends to infinity. Six models were trained, one for each of the best four-, three-, and two-muscle input sets described previously and for both the squared exponential and neural network covariance functions. The best window structure determined from the analysis described in section 1.III.C was used for each model. Since there were only six models to train and this was the last level of model characteristics explored, we allowed the hyperparameter optimization to continue until convergence.

### E. Muscle Activation Dynamics

One potential use of this method may be to estimate muscle force using a reduced sEMG array wherein excitations are input to a muscle activation model. To evaluate the error in estimating muscle activations in this way, we used estimated and measured excitations as inputs to a critically damped, linear, second-order activation model with unity gain and 3 Hz natural frequency. Only the four-muscle input set with squared exponential covariance function was used for this analysis.

### F. NNMF-Based Comparison

No previous study has presented similar results in estimating muscle excitations from a measured subset to which ours could be compared. However, previous work has shown that synergy excitations computed from a subset of measured muscles could reconstruct unmeasured excitations if the synergy weights for the unmeasured muscles were known. Following this approach, three synergy vectors and the corresponding excitations were determined for the best four-muscle input set using an iterative procedure utilizing both the multiplicative update and alternating least squares algorithms for non-negative matrix factorization (NNMF). This was done using test set data for each subject. Synergy weights for the output muscles were determined using linear least squares regression.

### G. Statistical Analysis

All models were evaluated using the performance metrics described in section 1.III.B: r, VAF, RMSE, and MAE. RMSE and MAE values are expressed as a percentage of MVC. These quantify estimation performance by comparing estimated muscle excitations to measured excitations in the test set (all unseen data). Metrics were computed for each output muscle separately and averaged across subjects. Average correlations were corrected using Fisher's z transformation and interpreted qualitatively as weak (r≤0.35), moderate (0.35<r≤0.67), strong (0.67<r≤0.90), and very strong (r≥0.90). Per the analysis described in section 1.III.E., activations driven by the estimated excitations were compared to those driven by the measured excitations using r, VAF, RMSE, and MAE. The reconstruction accuracy of the NNMF approach was compared to the estimation accuracy of the proposed method using VAF.

### 1.IV. Results

According to the z-score averaging method and following the analysis described in section 1.III.A, the best four-muscle input set was BF, PL, SOL, VL (r=0.76, VAF=86%, RMSE=3.6%, MAE=2.4%) and strong correlations (r>0.67) were observed for 200 out of the 210 sets. The best three-muscle input set was BF, PL, SOL (r=0.73, VAF=84%, RMSE=3.4%, and MAE=2.2%) and strong correlations were observed for 80 out of the 120 sets. The best two-muscle input set was LG, SOL (r=0.67, VAF=74%, RMSE=4.1%, and MAE=2.7%) and strong correlations were observed for only two out of the 45 sets (the other was PL, MG). These muscle sets were used for the remaining analyses.

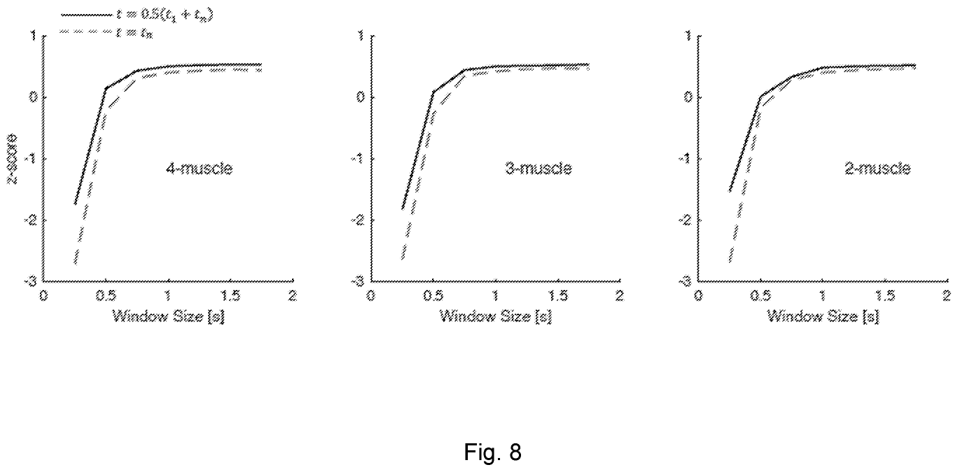

The window relative output time corresponding to half the window size, t=0.5(t₁+tₙ), performed better than t=tₙ for all models. Generally, larger window sizes performed better up to what appears to be a point of diminishing returns (FIG. 8). For both the four- and three-muscle input sets, the 1.75 and 1.50 s window sizes had a z-score of 0.53. For the two-muscle input set, the 1.75 s window size (z-score=0.52) was only slightly better than the 1.50 s window size (z-score=0.51). Thus, in the remaining analyses, a 1.50 s window size with 0.75 s window relative output time was used.

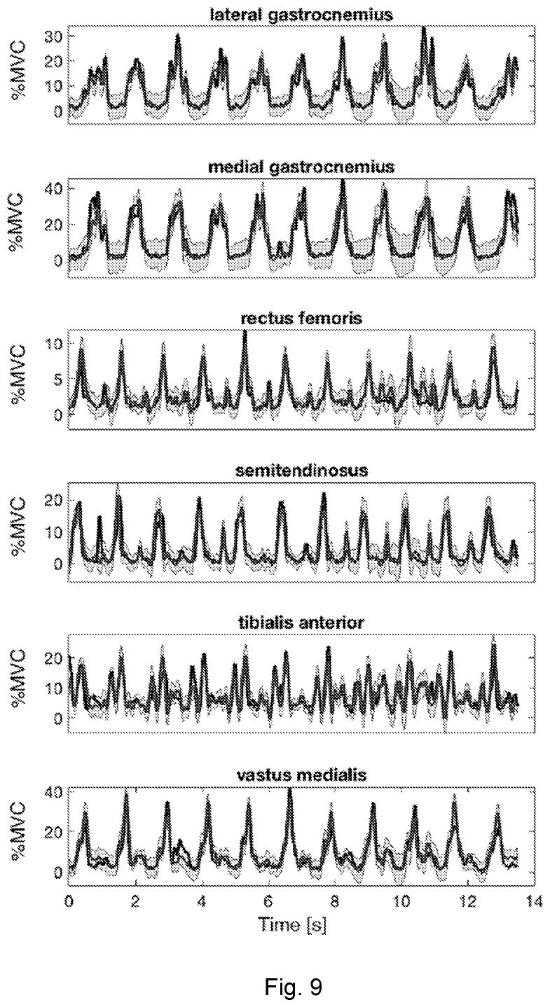

The estimation performance for models using the squared exponential and neural network covariance functions are presented in Table I. Although their performances were similar, the subject-averaged statistics shown in Table I suggest the stationary covariance function (squared exponential) performed better across all output muscles and input muscle sets except that the two-muscle input model with neural network covariance resulted in a slightly larger correlation (r=0.68) for the VL model compared to r=0.67 for the squared exponential covariance. For the four-muscle input set, all correlations were strong and on average accounted for ≥85% VAF. Likewise, all correlations were strong for the three-muscle input set except for the RF (r=0.66). The two-muscle input set performed surprisingly well with strong correlations across all muscles except moderate correlations observed for the BF (r=0.65) and RF (r=0.63). Further, the BF model accounted for only 55% VAF which was noticeably less compared to other muscles. The next lowest VAF for the two-muscle input set was 75% for ST while all models for the three-muscle input set explained more than 80% VAF. A graphical comparison of estimated and measured excitations is provided in FIG. 9.

TABLE I
Muscle Excitation Estimation Performance

| | | Stationary Covariance | | | | Non-Stationary Covariance | | | |
|---|---|---|---|---|---|---|---|---|---|
| Input | Output | r | VAF | RMSE | MAE | r | VAF | RMSE | MAE |
| 4 Muscles | MG | 0.89 | 88 (5) | 7.2 (2.2) | 4.7 (1.6) | 0.89 | 87 (5) | 7.4 (2.3) | 5.0 (1.7) |
| (BF, PL, SOL, VL) | LG | 0.81 | 85 (10) | 4.9 (1.9) | 3.2 (1.2) | 0.8 | 83 (13) | 5.1 (2.0) | 3.5 (1.5) |
| | TA | 0.81 | 88 (5) | 4.0 (2.1) | 2.8 (1.4) | 0.79 | 85 (7) | 4.3 (2.2) | 3.2 (1.7) |
| | ST | 0.82 | 82 (8) | 2.4 (1.4) | 1.5 (0.8) | 0.81 | 80 (10) | 2.5 (1.3) | 1.6 (0.8) |
| | VM | 0.78 | 87 (10) | 2.1 (1.3) | 1.3 (0.7) | 0.77 | 85 (11) | 2.2 (1.4) | 1.4 (0.8) |
| | RF | 0.68 | 88 (8) | 0.8 (0.3) | 0.5 (0.2) | 0.69 | 87 (10) | 0.8 (0.3) | 0.5 (0.2) |
| 3 Muscles | MG | 0.89 | 87 (5) | 7.2 (2.2) | 4.8 (1.6) | 0.89 | 87 (5) | 7.5 (2.3) | 5.1 (1.7) |
| (BF, PL, SOL) | LG | 0.8 | 84 (10) | 5.1 (2.1) | 3.3 (1.2) | 0.79 | 82 (12) | 5.3 (2.3) | 3.6 (1.6) |
| | TA | 0.8 | 87 (5) | 4.1 (2.1) | 2.8 (1.4) | 0.79 | 85 (6) | 4.4 (2.2) | 3.2 (1.7) |
| | ST | 0.81 | 81 (8) | 2.4 (1.4) | 1.5 (0.8) | 0.8 | 80 (8) | 2.5 (1.3) | 1.6 (0.8) |
| | VM | 0.76 | 83 (15) | 2.3 (1.5) | 1.4 (0.8) | 0.73 | 79 (19) | 2.5 (1.6) | 1.6 (0.9) |
| | VL | 0.73 | 84 (11) | 1.5 (1.2) | 0.9 (0.7) | 0.7 | 80 (16) | 1.7 (1.4) | 1.1 (0.9) |
| | RF | 0.66 | 86 (7) | 0.8 (0.3) | 0.5 (0.2) | 0.64 | 84 (10) | 0.9 (0.4) | 0.6 (0.2) |
| 2 Muscles | MG | 0.91 | 90 (3) | 6.5 (1.7) | 4.3 (1.3) | 0.91 | 90 (4) | 6.5 (1.7) | 4.4 (1.3) |
| (LG, SOL) | PL | 0.76 | 80 (8) | 6.5 (2.3) | 4.2 (1.4) | 0.76 | 78 (8) | 6.7 (2.3) | 4.5 (1.5) |
| | TA | 0.8 | 87 (3) | 4.1 (1.9) | 2.9 (1.3) | 0.78 | 85 (5) | 4.4 (2.1) | 3.2 (1.6) |
| | ST | 0.76 | 75 (10) | 2.7 (1.5) | 1.8 (0.9) | 0.74 | 73 (12) | 2.8 (1.5) | 2.0 (1.0) |
| | BF | 0.65 | 55 (29) | 4.8 (2.3) | 2.9 (1.4) | 0.61 | 46 (33) | 5.2 (2.3) | 3.4 (1.5) |
| | VM | 0.71 | 82 (15) | 2.4 (1.5) | 1.5 (0.8) | 0.69 | 79 (18) | 2.5 (1.6) | 1.6 (0.9) |
| | VL | 0.67 | 81 (12) | 1.5 (1.1) | 1.0 (0.7) | 0.68 | 81 (12) | 1.5 (1.1) | 1.0 (0.7) |
| | RF | 0.63 | 84 (11) | 0.9 (0.4) | 0.6 (0.3) | 0.63 | 83 (10) | 0.9 (0.3) | 0.6 (0.2) |

Measured and estimated excitations were compared for the best four, three, and two muscle input sets, the best input window structure, and for a stationary (squared exponential) and non-stationary (neural network) covariance function using only data from the test set. Performance metrics include correlation coefficient (r); percentage of variance accounted for (VAF); root mean square error (RMSE) in units percentage of MVC; mean absolute error (MAE) in units percentage of MVC. The reported VAF, RMSE, and MAE values are the average across all subjects for each muscle with the standard deviation in parentheses. The r values are the average correlation coefficients across all subjects using Fisher's z transformation as described by Silver and Dunlap (1987).

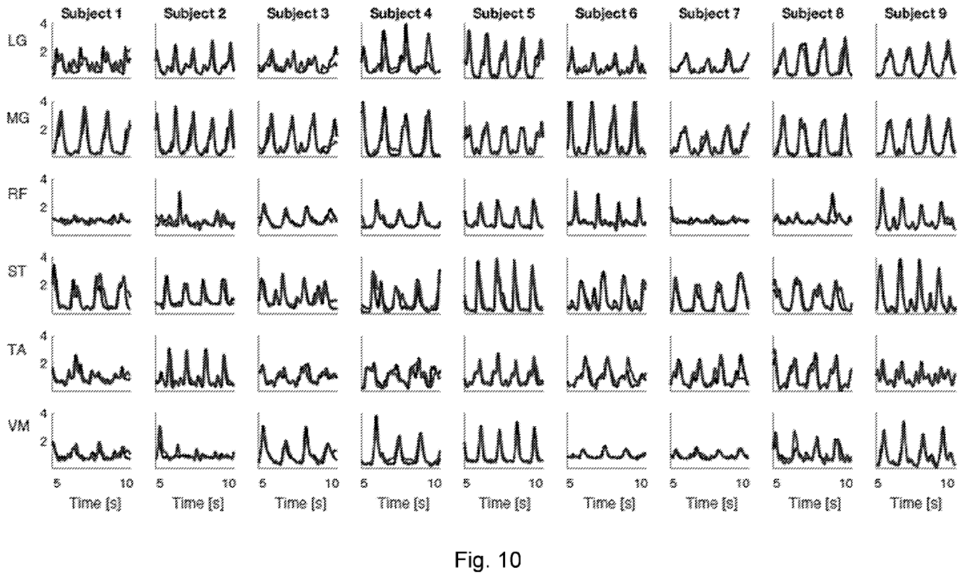

Comparison of muscle activations informed by measured and estimated excitations are presented in Table II (four-muscle input set). Strong to very strong correlations were observed across all muscles and on average, activations driven by the estimated excitations were able to explain more than 90% VAF in the true activation time-series except for the ST (VAF=89%) with MAE<4.0%. FIG. 10 depicts a graphical comparison of estimated and true activations for every output muscle and for every subject.

The NNMF-based reconstruction performance quantified by VAF was 74±15% for MG, 82±9% for LG, 66±13% for TA, 67±15% for ST, 89±9% for VM, and 80±15% for RF which was less than that observed for all output muscle models using the four-muscle input set except for the VM (NNMF: VM VAF=89%, GP: VM VAF=87%).

### 1.V. Discussion

In this section, we have demonstrated the ability to accurately estimate unmeasured muscle excitations using a subset of measured sEMG data. We introduced the notion of muscle synergy functions (FIG. 7) and developed from first principles the Gaussian process regression-based approximation to their behavior (section 1.II).

TABLE II
Muscle Activation Estimation Performance

| Muscle | r | VAF | RMSE | MAE |
|---|---|---|---|---|
| MG | 0.93 | 93 (4) | 5.2 (1.8) | 3.5 (1.2) |
| LG | 0.86 | 91 (6) | 3.5 (1.3) | 2.4 (0.9) |
| TA | 0.83 | 92 (5) | 3.1 (1.8) | 2.1 (1.3) |
| ST | 0.88 | 89 (6) | 1.7 (1.1) | 1.1 (0.6) |
| VM | 0.84 | 92 (5) | 1.5 (1.0) | 1.0 (0.6) |
| RF | 0.74 | 93 (5) | 0.5 (0.2) | 0.4 (0.1) |

Measured and estimated excitations from the test set for the four-muscle input set (BF, PL, SOL, VL) were used to estimate muscle activations. Performance metrics include correlation coefficient (r); percentage of variance accounted for (VAF); root mean square error (RMSE) in units percent MVC; mean absolute error (MAE) in units percent MVC. The reported VAF, RMSE, and MAE values are the average across all subjects for each muscle with the standard deviation in parentheses. The r values are the average correlation coefficients using Fisher's z transformation as described by Silver and Dunlap (1987).

An exhaustive search was used to study optimal input muscle sets. The set selection approach considered both the raw estimation performance metrics and weighted performance metrics according to the relative muscle PCSA. The former may be important in clinical applications where excitations are used to quantify motor control complexity and to monitor progression following knee surgery. Incorporating the additional PCSA-weighted average of performance metrics was done to purposely bias the muscle set selection such that the muscles with the greatest force producing capacity were estimated more accurately (on average). This choice was motivated by the potential use of this technique for estimating muscle force in remote gait analysis. This could be transformative for this area of research, providing insight into biomechanical variables more sensitive to disease etiology and that are ideal for personalizing rehabilitation (e.g., joint contact forces). Different selection criteria may be justified in other applications (e.g., muscle weights based on their relative contribution to the net joint moment during walking) and may lead to a different choice of an input muscle set. For example, to instrument a knee brace, one may wish to only consider muscles nearest the knee joint. In other applications where sensor placement is performed by the patient it may be beneficial to develop models using muscles which are easiest to locate for non-specialists. Other input muscle sets not further analyzed beyond the analysis described in section 1.III.A can be equally viable candidates. For example, 200/210 four-muscle and 80/120 three-muscle input sets achieved strong correlations in the estimation performance. Other input muscle sets may be used and are within the scope of the present disclosure.

The soleus was the only muscle consistently chosen across the four-, three-, and two-muscle input sets for the work in this section. This may be due to the PCSA-weighted averaging method as the soleus is the largest muscle and thus was given the largest weight (24%). The best four-muscle input set (BF, PL, SOL, VL) included a muscle that crossed the knee joint anteriorly and posteriorly and the posterior ankle but with no dorsiflexor. The only dorsiflexor measured in this study was the TA, however, in our analysis the TA was consistently well estimated suggesting its behavior is well-inferred from the other input muscles during walking.

A general increasing trend was observed between estimation performance and input window size. In section 1.II, we postulated that the input window size may serve to identify a neural control strategy to accomplish some sub-task during walking similar to how identified synergy vectors have been associated with certain phases of the gait cycle. The average stride time of individuals in this study was 1.31 s and thus, using the 1.50 s window size and 0.75 s window relative output time, the synergy functions were able to ‘see’ approximately the previous and future half gait cycle of input excitations to infer output excitations. As such, in some embodiments, optimal window size may be stride time (or gait speed) dependent.

We note that the final estimator in (10) and (12) truly captures the synergistic relationship between input and output muscles as it has no indication of time. Thus, the approximated synergy functions are not directly time dependent. Further, any gait phase dependent periodicity inherent in the sEMG signal during walking is not directly modeled (e.g., by including gait-phase as an input). For this reason, the GP model may be sensitive to step-by-step variation in both sEMG magnitude and frequency (FIG. 9). This is visually apparent for the TA in FIG. 9 (from a single subject).

In addition to time independency, the present model is also independent of any kinematic behavior which may be incorporated by including inertial sensor data (e.g., angular rate, acceleration) as additional inputs. In some embodiments, including a broad enough range of activities in the training set (e.g., stair ascension, cycling) may allow learning more generalizable phenomena enabling less strict activity identification specificity in the remote gait analysis pipeline (e.g., a locomotion activity classifier is less strict than a walking activity classifier).

The disclosed synergy function models can be modified in many ways beyond input muscle sets and input window structure, especially regarding the GP model (e.g., mean and covariance function). It would have been infeasible to exhaustively explore all covariance functions. Rather we explored a stationary and non-stationary covariance function in (16) and (17) respectively and found (negligible) superior performance using the squared exponential covariance. The squared exponential covariance utilized in this work was isotropic in that the characteristic length scale, θ₂ in (16), was the same for all inputs. In some embodiments, an alternate approach is to include length scales specific to each input muscle which can ultimately weight the relevance of specific input muscles for particular output muscles. For example, the VL may be especially relevant for estimating VM output excitations. Along these lines, a linear mean function may further improve estimation accuracy, μₘ(x)=ρ^{T}x, wherein input muscles which are anatomically and/or functionally similar to the particular output muscle can be preferentially weighted according to the associated element in ρ (the hyperparameters). Finally, although all estimated output muscle excitations were in the interval [0 1] (a physiological constraint) the conditional distribution characterized in (10) and (11) suggests nonzero probability densities for excitations outside this interval. For this reason, a beta likelihood function may be more appropriate.

We explored the potential of driving muscle activation dynamics given the estimated muscle excitations. Our results suggest this is a valid approach with MAE less than 4.0% across all output muscles, strong correlations, and an ability to explain between 89-93% VAF (Table II). For all output muscles, activation estimation performance metrics were greater than for excitation estimation. Activation dynamics essentially act to smooth input excitations which may underlie this observation. Thus, results may be further improved if a lesser low-pass filter cutoff frequency were chosen in the sEMG pre-processing. We chose 6 Hz as this is common for muscle force estimation, but some embodiments may use.

While there is no other study using a comparable approach (i.e., using only sEMG inputs) to compare our results, a comparison to a reconstruction approach using the more familiar NNMF-based synergy analysis is instructive. The present approach explained more percentage VAF across all output muscles except a negligible difference for the VM. The NNMF-based reconstruction VAF for some muscles was less than that reported for the three-synergy configuration in other work. One explanation is that synergies were extracted from only four muscles in this study compared to eight in the other work and the optimal subset for the NNMF reconstruction may be different than for the GP model. Further, the results in the other work were for only two subjects compared to nine in this study. When comparing the NNMF reconstruction and GP estimation, it is important to note that the NNMF technique has an unfair advantage in that the synergy model was informed partly by the data it sought to reconstruct. For this reason, the NNMF approach does not solve the problem of estimating unmeasured excitations using a measured subset; it only provides insight into the feasibility of doing so. This is in contrast to the proposed approach wherein the GP model was informed by a completely different dataset than it was tested on.

In some embodiments, a current limitation in using Gaussian process regression (or any non-parametric regressor) is computation time. For the four-muscle input set, the average total model training time (six total synergy models, one for each output muscle) was 31 minutes per subject. For the same configuration, the average computation time to estimate 1.0 s of data was 0.2 s. This is less of a burden for remote patient monitoring applications as current pipelines generate clinical reports offline after a full 24-hour recording. The estimation of muscle excitations in (12) requires inversion of the large (N×N) covariance matrix Σₘ. This inversion can be performed before deployment and thus speed up the estimation. The matrix Σₘ⁻¹ is also required for computing the estimation variance in (11) and (14). Modeling these variances are a benefit of the GP approach as they provide an indication of the uncertainty of the muscle excitation estimate which may be useful if excitations are allowed to be adjusted in a sensor fusion framework given other measurements of the musculoskeletal system dynamics.

### Section 2

### 2.I. Introduction

Remote patient monitoring, enabled by advances in wearable technology and algorithms for human movement analysis, promises to improve the assessment and treatment of musculoskeletal disease. Recent work quantifying stride-by-stride gait mechanics at segment-, joint-, and muscle-specific levels has shown that these variables may provide more sensitive measures of patient health than the more typical gross measures of physical activity. Despite these advances, many of the most clinically relevant variables have yet to be observed outside of controlled, laboratory environments. Ideally, assessments would quantify cumulative loading of muscle and articular tissue across every step taken in daily life to best characterize the mechanical stimuli driving tissue adaptation. Characterization at this level could enable personalized therapies and optimal evaluation of intervention efficacy. Further, remote monitoring of these variables could provide novel insight into musculoskeletal disease etiology. For example, in osteoarthritis, load is known to have a positive effect on healthy tissue and yet detrimental effects on diseased tissue. It is not known when this transition occurs, but monitoring cumulative tissue loads under free-living conditions could allow the investigation of these and other cumulative load-dependent phenomena. However, new methods are needed for characterizing joint and muscle mechanics in remote environments to enable these important clinical and research advancements.

The biomechanical variables associated with these analyses (e.g., joint moment, muscle force) provide far more clinical utility than what is typically evaluated remotely (e.g., physical activity, spatiotemporal gait variables). While both frontal and sagittal plane joint moment are important concerning musculoskeletal diseases of the knee, knee flexion moment (KFM) in particular, is thought to play an especially important role in early knee osteoarthritis and for monitoring patients following reconstructive surgery of the anterior cruciate ligament (ACLR). It may also be advantageous to characterize individual muscle function. Muscle power, for example, is a well-known determinant of physical function and the phenomena of work- and load-induced muscle hypertrophy motivate tracking cumulative muscle work and force which may provide a basis for optimal exercise prescription and understanding subsequent dose-response relationships. These analyses may be especially relevant for monitoring patients post-ACLR wherein the knee extensor and flexor musculature are compromised due to muscle atrophy and muscle activation deficits. In this case, early intervention is critical and continuous, remote monitoring augments personalized rehabilitation for targeting specific biomechanical outcomes.

While physics-based techniques exist for estimating these clinically relevant variables from wearables, they require complex sensor arrays that discourage use outside of research contexts. Regression algorithms have been proposed to reduce the number of required sensors, but at the expense of generalizability. Further, machine learning techniques do not characterize the dynamics of some relevant internal state variables (e.g., muscle contraction dynamics) which are modeled in physics-based techniques and may be particularly useful in the application of these techniques for rehabilitation. To leverage the strengths of both approaches, hybrid solutions have been proposed wherein machine learning is used only where the physics are not well understood or insufficiently informed. To the authors' knowledge, only one pilot study has explored a hybrid method wherein KFM was estimated in an electromyography (EMG)-driven simulation. However, validation was for a single subject and machine learning was used to solve for some kinematics that could have been estimated from physics-based techniques (e.g., knee flexion angle from thigh- and shank-worn IMUs).

In this section, we introduce another method for characterizing muscle and joint mechanics during walking which utilizes EMG-driven simulation of muscle contraction and inertial measurement unit (IMU)-driven forward kinematics. This approach was designed to enable more effective management of musculoskeletal diseases of the knee joint. We demonstrate the performance of the present approach for characterizing KFM as well as individual muscle moment and work by comparison to standard methods.

### 2.II. Present Technique

FIG. 1 summarizes the present technique—to model each muscle contributing to KFM as a Hill-type actuator and simulate contraction using an EMG-driven approach. The required inputs then are the excitation of each muscle and the length of each muscle-tendon unit (MTU). The novelty of the current work is to implement this approach with a reduced sensor array such that the technique may be feasibly deployed for remote monitoring. To this end, we used only two IMUs (one each on the thigh and shank) to solve for the system kinematics and compute MTU lengths in a process referred to as inertial motion capture (IMC). Further, the number of required electrodes was reduced by instrumenting only a subset of muscles whereupon the remaining excitation signals (would be unmeasured in practice) are informed by the measured subset using a Gaussian Process (GP) model of the associated muscle synergy functions. In the current implementation, the subset included three muscles: medial (MG) and lateral (LG) gastrocnemius and vastus medialis (VM). These locations were chosen because they are close to the knee joint and enable instrumentation of a knee brace for practical deployment.

The musculoskeletal model, Hill-type muscle models, and GP models all require a one-time calibration. Calibration of a sensor-to-segment model and EMG normalization is required each time the IMUs and electrodes are attached (e.g., daily). The following sections describe these models and their calibration as well as the IMC analysis, EMG-driven simulation of muscle contraction, and the computation of the biomechanical variables of interest.

### A. Musculoskeletal Model and Calibration

The musculoskeletal model consisted of five segments including a foot, shank, thigh, patella, and pelvis; three joints including a two degree-of-freedom (DOF) ankle, single DOF knee (tibiofemoral), and a three DOF hip; and ten MTUs including the MG, LG, VM, vastus intermedius (VI), vastus lateralis (VL), rectus femoris (RF), long (BFL) and short (BFS) heads of the biceps femoris, semitendinosus (ST), and semimembranosus (SM). MTU path points (origin, insertion, and via points), cylindrical geometry of the femoral condyle, and the origin and insertion of the patellar ligament were taken from other work. Average path points were used for multi-element muscles (e.g., superior and inferior elements of the VL). A single via point was used for the MG and LG located at the apex of the shortest curve between origin and insertion points wrapping posteriorly around the cylinder modeling the femoral condyle. The model had 12 mechanical DOFs described by 28 generalized coordinates including translational (n=3) and rotational (n=4, quaternion) coordinates for each segment except the patella (configuration was a function of the knee flexion angle) subject to 16 equality constraints: four enforce the quaternion unit length constraints; nine enforce the non-translating joint constraints; and three enforce the hinge and universal joint constraints on the knee and ankle. The angle of the patella relative to the patellar ligament was modeled as a constant 20°. The angle of the patellar ligament (constant length) relative to the shank was based on the results of other work.

Two calibration trials were used to calibrate the subject-specific musculoskeletal model. A static calibration (standing in anatomical position) was used as the reference configuration in which relative positions of segment-fixed points define each rigid body segment including markers and MTU path points. A functional calibration trial was used to identify joint centers and the knee flexion axis. The subject exercised multiple movements of each joint exciting all joint DOFs through the full range of motion. In this case, segment kinematics were computed independently without regard for any mechanical constraints: orientation via Davenport's solution to Wahba's problem wherein every unique two-marker combination for all segment-fixed markers during the static calibration trial were used as the reference vectors (weighted by their length squared) and position as done by others. Hip and ankle joint centers were estimated using the pivoting algorithm and the knee flexion axis using the SARA method. Knee joint center was defined as the point on the knee flexion axis closest to the femoral epicondyle midpoint.

Segment-fixed coordinate systems were constructed with basis vectors coincident with the principal axes of inertia and origin with the segment center of mass (inertial parameters taken from other work). Local MTU path points were scaled based on an anthropometric measure of each segment relative to the same from the data reported in other work (e.g., segment length). Knee extensor and medial hamstring insertion points were adjusted to better align their knee flexion moment arms with published data.

### B. Sensor-to-Segment Model Calibration

The daily sensor-to-segment model calibration requires three calibration trials: the same static and functional (hip/knee joint) calibration trials used for calibrating the musculoskeletal model and straight walking. The system configuration during the daily static calibration is assumed equivalent to the reference configuration. The TRIAD algorithm was used to determine the orientation of the IMU frames relative to the segment frames. Reference vectors were the knee joint flexion axis and gravity vector with full trust given to the former. The representation of these vectors in the segment frames were taken from the reference configuration. The representation of the gravity vector in each IMU frame was computed as the average direction of the accelerometer output during the static calibration trial. The representation of the knee joint flexion axis in each IMU frame and the position of the knee joint center relative to each IMU were determined using a nonlinear least-squares method. Data recorded during the hip and knee joint movements of the daily functional calibration trial were used for both calibrations (knee joint flexion axis and knee joint center) in addition to the walking calibration trials for calibrating the knee joint flexion axis.

### C. Inertial Motion Capture

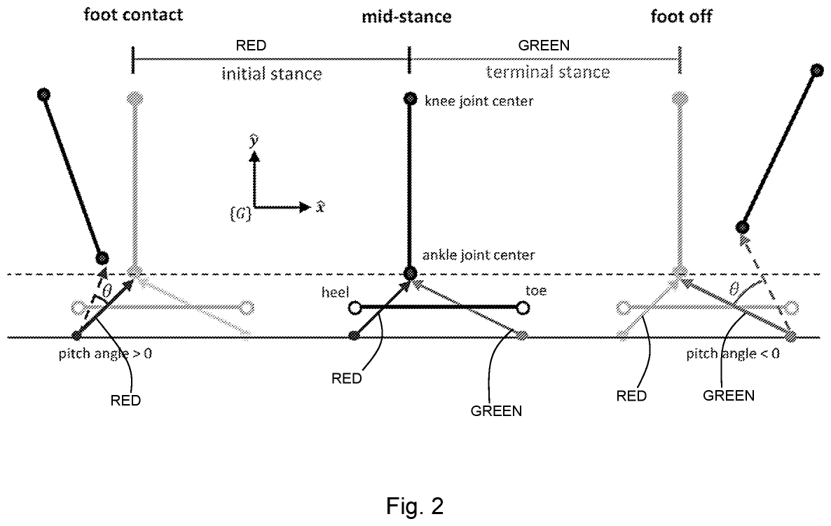

Shank and thigh IMU data were first expressed relative to their segment coordinate systems based on the calibrated sensor-to-segment model. The medio-lateral component of the shank gyroscope signal was used to identify foot contact and foot off events. Shank accelerometer data were used to identify the most still quarter of the identified stance phase (interval for which the sum of the accelerometer signal variances was least) during which the average acceleration was used to estimate shank orientation at the middle of the interval (assuming zero heading angle). Shank orientation before and after this mid-stance instant was obtained using the analytic solution to the quaternion kinematic equation following an assumption of constant angular rate between measured samples equal to the average of the two samples. Knee flexion angle (θ) was estimated using an RTS Kalman smoother implementation of a complementary filter. Thigh orientation was determined from shank orientation and knee flexion angle. Pelvis orientation was assumed neutral except that the heading angle was constant and equal to the average shank heading angle during stance. The acceleration of the knee and ankle joint centers was computed from shank accelerometer data (after removing gravity) along with shank gyroscope-measured angular velocity and the known joint center position relative to the shank IMU. Ankle joint center position was computed by double (trapezoidal) integration of ankle joint center acceleration. Foot heading angle was equivalent to that of the shank, roll angle was zero, and pitch angle was computed based on a simple foot-ground contact model (FIG. 2). Given all segment orientations and the ankle joint center position throughout stance phase, the remaining generalized coordinates were given from the calibrated musculoskeletal model. MTU length and knee flexion moment arm were computed as in other work.

### D. EMG-Driven Simulation of Muscle Contraction

MTU geometry was modeled such that pennation angle (ϕ) and tendon length ( [symbol] _{T}) were explicit functions of MTU ( [symbol] _{MTU}) and muscle fiber ( [symbol] ₘ) length as per

$$ϕ = a sin ( \frac{{ℓ}_{0} sin {ϕ}_{0}}{{ℓ}_{m}} ) \qquad ( 18 )$$

$${ℓ}_{T} = {ℓ}_{MTU} - s \qquad ( 19 )$$

where s (equal to the product [symbol] ₘ cos ϕ) is the projection of the fiber length onto the MTU, [symbol] ₀ is the optimal fiber length, and ϕ₀ is the pennation angle when [symbol] ₘ= [symbol] ₀. Tendon force (ƒ_{T}) was modeled as per

$${f}_{T} = {f}_{m}^{0} μ ( exp ( E {ϵ}_{T} ) - 1 ) \qquad ( 20 )$$

where ƒₘ⁰ is the maximal isometric force of the muscle, μ is constant and equal to exp(−0.04E), ϵ_{T} is the tendon strain modeled as a function of [symbol] _{T} and tendon slack length ( [symbol] ₛ), and the parameter E (df_{T}/dϵ_{T} when ϵ_{T}=4%) was set to 35.00. Muscle force projected onto the ƒ_{T} line of action (ƒₘ) was modeled as per

$${f}_{m} = {f}_{m}^{0} ( {f}_{v} {f}_{ℓ} {f}_{A} + {f}_{p} + β {\tilde{v}}_{m} ) cos ϕ . \qquad ( 21 )$$

The parenthesized term in (21) is the normalized muscle force which is scaled by ƒₘ⁰ and projected onto the ƒ_{T} line of action via multiplication with cos 0. The functions ƒᵥ, ƒ [symbol] , and ƒₚ model the force-velocity, active force-length, and passive force-length properties of muscle, respectively. Fiber length [symbol] ₘ normalized by [symbol] ₀ is input to both ƒ [symbol] and ƒₚ. The input to ƒᵥ is fiber velocity (vₘ) normalized by the maximal fiber shortening velocity (set to 15 optimal fiber lengths per second); denoted \tilde{v}ₘ. In this study, ƒᵥ and ƒ [symbol] were both modeled as in other work and ƒₚ as in other work with passive muscle strain due to maximal isometric force (ϵ₀^{m}) set to 0.55. The input to the activation nonlinearity function (ƒ_{A}), modeling the nonlinear relationship between activation and muscle force, is the activation signal (α), the dynamics (\dot{α}) of which were driven by the muscle excitation signal (e). Note that \dot{α} is a function of α, e, and other parameters (e.g., time constants) modeling the activation dynamics (see section 2.II.G and online supplementary material for details). The output of ƒ_{A} is also used in ƒ [symbol] to model the dependency of the optimal fiber length on muscle activation. The product β\tilde{v}ₘ models damping effects within the fiber where the coefficient (β) was set to 0.01. Several scalar parameters (e.g., ƒₘ⁰, [symbol] ₀, [symbol] ₛ) and functions (ƒ_{A}, \dot{α}) not yet specified were determined in a calibration process described in section 2.II.G.

In the current embodiment, fiber length [symbol] ₘ and muscle activation α were the state variables for the muscle contraction dynamics and system inputs were MTU length [symbol] _{MTU} and muscle excitation e. To compute muscle excitation e, raw EMG were digitally high-pass filtered at 30 Hz, rectified, low-pass filtered at 6 Hz and normalized by the maximal value across several activities (e.g., maximal isometric contractions, walking, running). All filters were 4^{th} order, zero-phase, Butterworth filters with double-pass adjustments. The excitation of the BFS, SM, and VI was assumed equivalent to that of the BFL, ST, and the average of VM and VL, respectively. The equivalence between the tendon (ƒ_{T}) and muscle (ƒₘ) force gives rise to the equilibrium equation

$${f}_{m} - {f}_{T} = 0 \qquad ( 22 )$$

which is an implicit formulation for the dynamics of the fiber length state variable. An implicit solver (ode15i in Matlab) was used to numerically integrate (22). Initial conditions for [symbol] ₘ and vₘ consistent with (22) were found numerically (decic in Matlab). This required an initial guess that may not satisfy (22) for which a rigid tendon was assumed. Activation dynamics were simulated using a Runge-Kutta formula (ode45 in Matlab).

### E. Computation of the Biomechanical Variables of Interest

Net KFM was computed as the sum of the flexion moments generated by each MTU given by the product of ƒₘ and the knee flexion moment arm. Cumulative concentric (W_{con}) and eccentric (W_{ecc}) work were computed using a trapezoidal approximation to the line integral

$${W}_{k} = \int \vert {f}_{k} ds \vert \qquad ( 23 )$$

for k∈{ecc, con} where |⋅| denotes absolute value and

$${f}_{con} = \{ \begin{matrix}{f}_{m} , & ds < 0 \\ 0 , & otherwise\end{matrix} \qquad ( 24 )$$

$${f}_{ecc} = \{ \begin{matrix}{f}_{m} , & ds > 0 \\ 0 , & otherwise\end{matrix} . \qquad ( 25 )$$

### F. Gaussian Process Model Calibration

The present technique uses excitations computed from the raw EMG of the three instrumented muscles to estimate the other four (unmeasured in practice). To this end, four GP models were trained (using open-source toolboxes) to approximate the four associated muscle synergy functions. Input muscles were VM, MG, and LG. Input window length was 1.50 s with a 0.75 s window relative output time. The GP model covariance function was the isotropic squared exponential and the mean function was constant.

### G. Hill-Type Muscle Model Calibration

A set of calibration walking trials from which a ground truth estimate of KFM based on inverse dynamics (ID) and a reference EMG-driven (referred to as OMC-Full) estimate were required for identifying Hill model parameters. Biomechanical variables were computed via OMC-Full in the same way as for the proposed technique (referred to as IMC-GP) except kinematics were solved using optical motion capture (OMC, described below) and a full set of measured excitations as opposed to the three-muscle subset.

### 1) Inverse Dynamics

The 28 generalized coordinates were found by minimizing a squared-error objective (errors between model-based and measured marker positions) subject to the 16 mechanical constraints described previously. The optimal solution was found using the interior-point algorithm (fmincon in Matlab) with analytic Jacobian and Hessian matrices of the objective and constraint equations. The constraint tolerance was set to 1e-6 and all markers were weighted equally. Segment linear and angular velocities and accelerations were approximated using the 5-point central difference method (quaternion velocities were computed first and mapped to angular velocities) and were low-pass filtered using a 4^{th} order, zero-phase, Butterworth filter (6 Hz cutoff frequency) with double-pass adjustments. The knee flexion moment arm and length of each MTU were computed as for the IMC analysis. Intersegmental forces and moments were computed using the recursive Newton-Euler algorithm and KFM by projecting knee intersegmental moment onto the flexion axis.

### 2) Parameter Optimization

Several physiological parameters related to the contraction dynamics may advantageously be optimized for each person and muscle (usually via global optimization). A novelty of the present work is the inclusion of categorical parameters in the tunable parameter set. Specifically, we used Bayesian optimization to optimize two functions, the activation nonlinearity function ƒ_{A} and the model of the activation dynamics \dot{α}, in addition to five scalar parameters. Optimal fiber length [symbol] ₀ and tendon slack length es are often included in the tunable parameter set. However, to prevent overfitting we chose to reduce the number of tunable parameters (which would otherwise be larger by inclusion of ƒ_{A} and \dot{α}) by removing [symbol] ₀ and [symbol] ₛ. Instead, [symbol] ₀ and [symbol] ₛ were optimized similar to previous work so that the range of [symbol] ₘ normalized by [symbol] ₀ during walking gait would be within the range ( [symbol] _{m,min}, [symbol] _{m,max}) of published data if a rigid tendon model were used. In this case,

$$[ \begin{matrix}{ℓ}_{MTU , max} \\ {ℓ}_{MTU , min}\end{matrix} ] = [ \begin{matrix}1 & {\tilde{ℓ}}_{m , max} cos {ϕ}_{max} \\ 1 & {\tilde{ℓ}}_{m , min} cos {ϕ}_{min}\end{matrix} ] [ \begin{matrix}{ℓ}_{s} \\ {ℓ}_{0}\end{matrix} ] \qquad ( 26 )$$

where ϕₖ=asin( [symbol] _{m,k}⁻¹ sin ϕ₀) for k∈{max, min} and the range of MTU lengths ( [symbol] _{MTU,min}, [symbol] _{MTU,max}) were subject-specific and taken from the walking calibration trials. The solution to (26) yields the optimal [symbol] ₀ and [symbol] ₛ.

Bayesian optimization was used to tune a scalar that scaled the maximal isometric force ƒₘ⁰ and five muscle activation parameters: activation dynamics model \dot{α}, activation time constant τₐ, activation-to-deactivation ratio τₐ/τ_{d} where τ_{d} is the deactivation time constant, activation nonlinearity function ƒ_{A}, and a parameter in ƒ_{A}. Muscles were grouped such that those belonging to the same group were assumed to have the same properties. Similar to previous work, three groups were permitted based on MTU structure and function: knee extensors, hamstrings, and gastrocnemii. Further, due to the association between fiber type distribution and the activation-force relationship, we required muscles in the same group to have a similar proportion of type I (slow-oxidative) fibers. All muscles within the three groups described previously with this fiber type proportion less than 60% were placed in a new group as were those greater than or equal to 60%. This was the case only for the hamstrings where SM and ST were both 50% type I, while BFL and BFS were both 65%. Thus, the four muscle groups were the knee extensors (VL, VM, VI, RF), lateral (BFL, BFS) and medial (SM, ST) hamstrings, and gastrocnemii (MG, LG) yielding 24 total tunable parameters.

The strength scalar (range: 0.5-2.0) scaled ƒₘ⁰ initialized as the product of the physiological cross-sectional area and the muscle stress when ƒₘ=ƒₘ⁰ (set to 0.30 MPa). Five activation dynamics models were considered (see online supplementary material for details): (1) a 1^{st} order, linear model, (2) a 1^{st} order, nonlinear, piecewise-continuous model, (3) a 1^{st} order, bilinear model, (4) a 2^{nd} order, linear model, and (5) a piecewise version of model (4). All models were unity gain with an electromechanical time delay (40 ms). The range of τₐ was 10-60 ms and of the ratio τₐ/τ_{d} was 0.25-1.00. Three functions were considered for ƒ_{A} (see online supplementary material for details): (1) an exponential model, (2) the A-model, and (3) the twice differentiable A-model.

The objective function was the average normalized mean squared error between the ID and OMC-Full estimate of KFM across all calibration walking trials where normalization was by the variance of the ID estimate. Optimization was executed using bayesopt in Matlab with the expected-improvement-plus acquisition function, 0.5 exploration ratio, 96 seed points (four times the number of parameters), 300 GP active set size, and the number of maximum objective function evaluations was set to 576 (the number of parameters squared).

### 2.III. Experimental Validation

### A. Data Collection

IMC-GP was validated on nine unimpaired subjects (four female, age: 21±1 years, height: 1.77±0.11 m, mass: 72.10±12.30 kg). Each subject performed 10 overground walking trials at a self-selected normal speed (1.36±0.20 m/s) for which the right foot completely contacted the force plate for a single contact. Thus, one stance phase for the right leg was analyzed per trial. Marker position data were captured using 19 cameras (Vicon Motion Systems, Oxford, UK, 100 Hz). Force plate (AMTI, Watertown, MA, USA) and raw EMG (BioStamp, MC10, Inc., Cambridge, MA, USA) data were collected at 1000 Hz. Electrodes were placed over the MG, LG, VM, VL, RF, ST, and BFL according to SENIAM recommendations. Force plate data were downsampled to 100 Hz for synchronization with marker data as were EMG data after excitations were computed. Shank and thigh IMUs (BioStamp, MC10, Inc., Cambridge, MA, USA, gyroscope range: ±2000°/s, accelerometer range: ±16 g, 250 Hz) were placed over the distal lateral shank and anterior thigh, respectively. IMU data were downsampled to 100 Hz and time synchronized with marker position data. Time synchronization was via cross-correlation of angular rate magnitude from the shank-worn gyroscope signal with the same from a separate shank-worn gyroscope (Opal, APDM Wearable Technologies Inc, Portland, OR, USA) that was hardware synchronized with the camera system. All subjects provided written consent to participate, and all activities were approved by the University of Vermont Institutional Review Board (#18-0518).

Each subject performed the static and functional calibration trials described previously for calibrating the musculoskeletal model. The first seven overground walking trials were set apart for calibrating the MTU parameters and GP models and the last three (test trials) were set apart for validation. The sensor-to-segment model calibration trials were the same static and functional calibration trials used for calibrating the musculoskeletal model and the test trials were used as the walking calibration trials. This mimics how calibration would be done in practice: the identified walking activity being evaluated would also be available for calibration.

### B. Statistical Analysis

Statistics characterizing performance of IMC-GP are reported only for the three test trials. Net KFM from IMC-GP was compared with both ID and OMC-Full and individual muscle moment was compared between IMC-GP and OMC-Full using Pearson's correlation coefficient (r) and root mean square error (RMSE). RMSE was expressed as a percentage of the average range of the two time-series being compared (denoted % range) and of the product of subject body weight (in N) and height (in m); denoted % BW·H. To compare our results to previous work, r and RMSE were computed for every time-series and then averaged. Average correlations were corrected using Fisher's z transformation and interpreted as weak (r≤0.35), moderate (0.35<r≤0.67), strong (0.67<r≤0.90), and excellent (r>0.90). Peak knee extension moment (KEM) during initial stance was compared between IMC-GP and ID using r, mean absolute error (MAE), and Bland-Altman analysis: mean error (ME) and 95% limits of agreement (LOA) with compensation for repeated measures. Pearson's r was used to evaluate the sensitivity of the IMC-GP analysis to variation in muscle work across subjects by comparison to the OMC-Full analysis. Work was considered only for the contraction type (eccentric or concentric) in which each muscle did the most work.

### 2.IV. Results

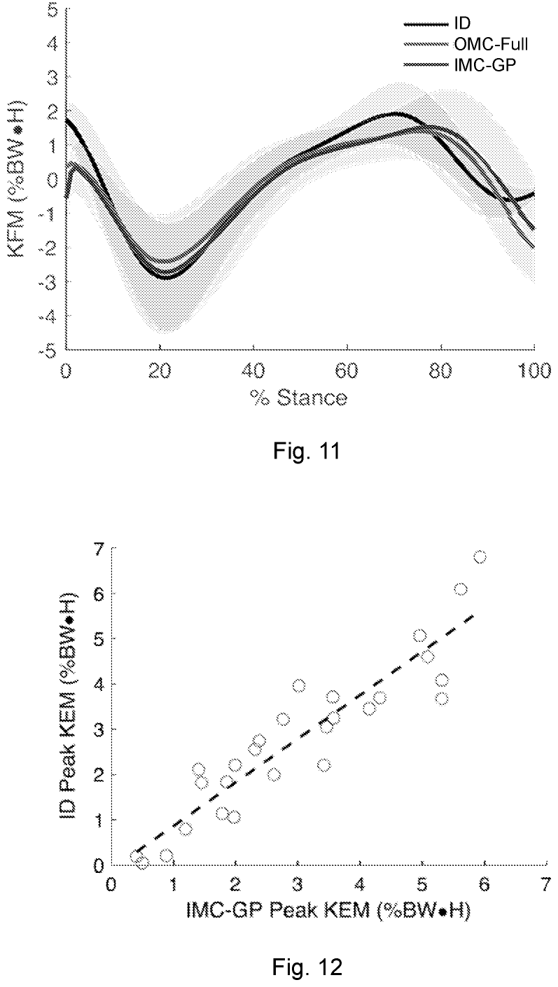

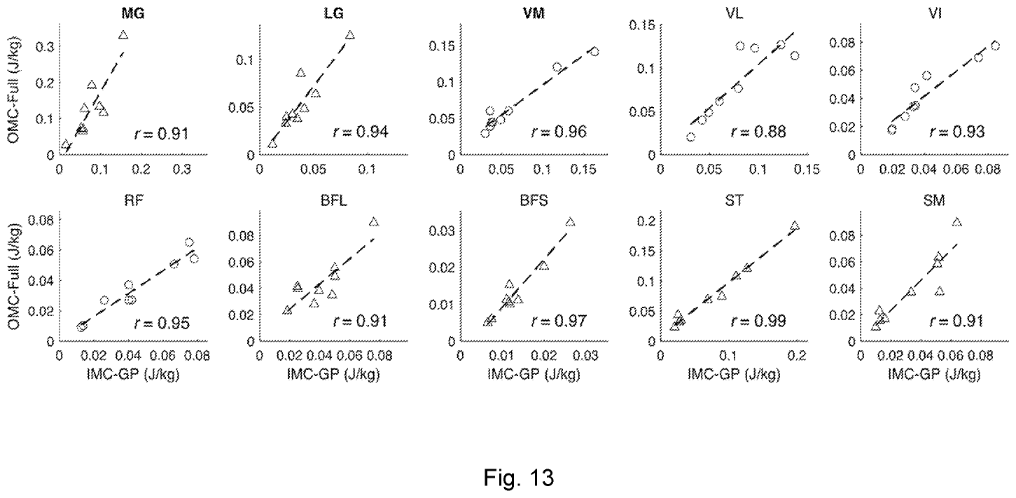

All three techniques yielded the same general trend in the KFM time-series (FIG. 11). This was supported statistically by strong to excellent correlations between IMC-GP estimates with those from ID (range: r=0.68-0.96, average: r=0.87) and OMC-Full (range: r=0.74-1.00, average: r=0.95) with RMSE less than 1.00% BW·H (Table I). Correlations between IMC-GP and OMC-Full estimates of individual muscle moment (see online supplementary material for a graphical comparison) were strong to excellent (r=0.81-0.99) across all muscles with RMSE between 6.46-26.33% range (Table I). Peak KEM was estimated to within 0.57% BW·H MAE of the ID estimate (ME: −0.22% BW·H, LOA: −1.54 to 1.11% BW·H) with excellent (r=0.92) correlation (FIG. 12). Excellent correlations were also observed between IMC-GP and OMC-Full estimates of cumulative muscle work across all muscles (FIG. 13) except for the VL (r=0.88).

### 2.V. Discussion

The most promising result from this validation was the estimation of KFM with strong correlations (r=0.87) and low errors (0.91% BW·H, 18.25% range RMSE) using IMC-GP. These results compare favorably with the current state-of-the-art in physics-based, wearables-only techniques. For example, Karatsidis et al. present an IMU-driven inverse dynamics analysis (17 IMUs, 17 segments) and optimization-based muscle force estimation. KFM was estimated for 11 healthy men across three walking speeds with a moderate correlation (r=0.58) and 1.9% BW·H (29.8% range) RMSE. Dorschky et al. present an approach based on optimal control of a musculoskeletal model wherein state variables tracked measured sensor signals (seven IMUs, seven segments) via trajectory optimization. KFM (full gait cycle) was estimated for 10 healthy men across three walking speeds with strong correlations (r=0.81) and 1.5% BW·H (27.1% range) RMSE. Compared to these methods, the present technique provides a significant reduction in sensor array complexity with comparable estimation performance.

The present technique also compares well with machine learning techniques. For example, estimation of KFM from the proposed technique was comparable to a neural network (NN) using EMG inputs (r²=0.81 vs. 0.76 for IMC-GP) and a linear model using data from an instrumented insole (p=0.89). In more recent developments, NN-based architectures with IMU inputs have been used to estimate KFM with 1.14% BW·H RMSE and r=0.98 in a four-sensor, four-segment configuration and with 18.4% range RMSE and r=0.72 in a two-sensor, two-segment configuration. In addition to characterization of KFM, IMC-GP provides complementary insight into the function and loading of individual muscles which are not modeled in machine learning techniques.

TABLE I
Estimation of Net KFM and Individual Muscle Moment

| | | | RMSE | RMSE |
|---|---|---|---|---|
| | | r | (% BW · H) | (% range) |
| Muscle | ID | 0.87 | 0.91 (0.31) | 18.25 (3.88) |
| Contributions | OMC-Full | 0.95 | 0.59 (0.30) | 12.86 (5.01) |
| | MG | 0.98 | 0.14 (0.13) | 10.02 (5.70) |
| | LG | 0.96 | 0.06 (0.05) | 13.09 (6.54) |
| | VM | 0.99 | 0.08 (0.05) | 6.46 (3.31) |
| | VL | 0.96 | 0.23 (0.14) | 13.56 (5.85) |
| | VI | 0.98 | 0.08 (0.05) | 9.83 (4.49) |
| | RF | 0.90 | 0.09 (0.05) | 20.63 (7.88) |
| | BFL | 0.92 | 0.08 (0.04) | 18.18 (6.97) |
| | BFS | 0.81 | 0.04 (0.02) | 26.33 (12.53) |
| | ST | 0.83 | 0.14 (0.09) | 24.65 (10.28) |
| | SM | 0.87 | 0.10 (0.06) | 21.02 (8.51) |

Bold entries indicate muscles for which measured excitations were used to simulate contraction whereas the others were based on the GP synergy function models. Parenthesized values are standard deviations.

One study has presented results for a hybrid approach similar to IMC-GP: machine learning informed both MTU kinematics and the mapping from an EMG subset to a full set. KFM (full gait cycle) was estimated with 26, 30, and 26% range RMSE for walking at 1.5, 3.0, and 5.0 kₘ/h, respectively, compared to 18.25% range RMSE in the current study.

Peak KEM during initial stance was estimated to within 0.57% BW·H MAE (i.e., 9.98% BM, percentage body mass) which is less than observed inter-limb differences for patients post-ACLR (1.08% BW·H), differences between patients post-ACLR and healthy controls (17% BM, 1.72% BW·H, 1.10% BW·H for patellar tendon graft), and gender differences observed for patients 12 months post-ACLR (13% BM). Therefore, the proposed technique appears able to detect clinically meaningful differences. From at least one study, the observed LOA and 9.98% BM MAE may appear too large to observe differences pre- and post-ACLR. Nevertheless, the observed excellent correlation (r=0.92, FIG. 12) suggests the present technique is sensitive to variation in peak KEM and thus could track patient recovery post-ACLR.

The present technique is dependent on EMG-driven simulation of muscle contraction and thus the OMC-Full analysis represents the theoretical ceiling of performance. Our comparison of IMC-GP to OMC-Full supports the proposed technique as a promising surrogate for EMG-driven analyses in remote monitoring applications (Table I). Estimation was best for the instrumented muscles (VM, MG, and LG in the current implementation). In a post-hoc comparison, we found that accurate estimation of individual muscle moment was due in large part to accurate estimation of muscle activation (from the GP models) based on similar RMSE (% range) of the two signals: 13.41 in activation RMSE vs. 13.56 in KFM RMSE for VL, 17.25 vs. 20.63 for RF, 25.63 vs. 24.65 for ST, and 25.81 vs. 18.18 for BFL. For instrumented muscles, estimation was better for VM than for MG and LG (Table I). IMU-driven forward kinematics likely explain this discrepancy wherein estimation of knee flexion angle (r=0.98, 4.080 RMSE) was better than for ankle dorsiflexion (r=0.53, 9.930 RMSE). Knee flexion angle was estimated using a Kalman smoother implementation of a previously validated complementary filter. However, to avoid the need for a foot-worn sensor, ankle dorsiflexion was given following a simple foot-ground contact model (FIG. 2). A more complex contact model (e.g., including toes) and/or a data fusion approach (e.g., fusing contact model estimates with a forward dynamic estimate driven by ankle muscles) may improve estimation. Still, the strong correlations and relatively low errors motivate use of IMC-GP for evaluating relative muscle contributions to KFM which has clinical implications for managing musculoskeletal disease.

The results of the correlation analysis suggest the proposed technique was sensitive to variation in muscle work from the reference EMG-driven analysis (FIG. 13). Muscle work is a known stimulus for hypertrophy and objectively quantifies exercise intensity. Thus, the present technique points toward continuous monitoring of individual muscle loading in daily life. This could be transformative for personalized therapy enabling novel patient profiling (e.g., characterizing patient-specific exercise dose-response relationships over time), evaluation of intervention efficacy, and the potential to adapt loading prescriptions for managing tissue over- and under-loading. While we demonstrated the characterization of individual muscle function via quantification of moment and work, other variables are necessarily characterized including joint, MTU, and muscle kinematics, power, and force. This thorough characterization of the musculoskeletal system is due to the physics-based nature of the proposed approach and is nonexistent in machine learning alternatives. For the latter, all desired outcome variables are modeled separately and physical relationships between inputs and outputs are not necessarily maintained.

### 2.VI. Conclusion

This section presents a hybrid machine learning- and physics-based technique for analysis of muscle and joint mechanics during walking using only wearable sensor data. Machine learning was used to reduce the number of required surface electrodes for EMG-driven simulation while data from two IMUs drove the system kinematics via physics-based techniques. This technique performed well compared to inverse dynamics and EMG-driven analyses with comparable performance to other wearables-only techniques with more complex sensor arrays. Further, it may easily be generalized for analysis of other muscles and joints not described herein. Importantly, the present technique allows sensor placement near the knee joint such that they could be integrated into a knee brace or sleeve for practical deployment.

Although the present disclosure has been described with respect to one or more particular embodiments, it will be understood that other embodiments of the present disclosure may be made without departing from the spirit and scope of the present disclosure.

1. A system for determining dynamics of a joint of an individual, comprising:
a first muscle contraction sensor configured to measure an excitation of a first muscle adjacent to a joint;
a first movement sensor configured to measure movement on a first side of the joint;
a second muscle contraction sensor configured to measure an excitation of a second muscle located adjacent to the joint;
a second movement sensor configured to measure movement on a second side of the joint;
a machine learning processor trained to determine a set of excitation values based on an excitation value from the first muscle contraction sensor and an excitation value from the second muscle contraction sensor, wherein the set of excitation values includes an excitation value for each muscle of the joint; and
a processor configured to determine a joint moment based on values from the first and second movement sensors and the set of excitation values from the machine learning processor.

2. The system of claim 1, wherein each of the first muscle contraction sensor and/or the second muscle contraction sensor is an electromyography (EMG) sensor.

3. The system of claim 1, wherein each of the first movement sensor and/or the second movement sensor is configured to measure movement in at least six degrees of freedom.

4. The system of claim 1, wherein each of the first movement sensor and/or the second movement sensor is an inertial measurement unit (IMU).

5. The system of claim 4, wherein each IMU comprises at least one gyroscope and at least one accelerometer.

6. The system of claim 1, wherein the machine learning processor has been trained by the individual performing a set training motions.

7. The system of claim 1, wherein the machine learning processor has been trained using movement data from a plurality of individuals.

8. The system of claim 1, wherein the processor is further configured to determine a set of muscle-tendon unit (MTU) lengths and moment arm values from the movement sensor values.

9. The system of claim 8, wherein the processor is further configured to determine a set of muscle forces based on the MTU lengths and muscle activation dynamics derived from the set of excitation values determined by the machine learning processor.

10. The system of claim 9, wherein the joint moment is determined based on the set of muscle forces and moment arm values.

11. The system of claim 1, further comprising a third muscle contraction sensor configured to measure an excitation of a third muscle located adjacent to the joint; and wherein the machine learning processor uses an excitation value from the third muscle contraction sensor in determining the set of excitation values.

12. The system of claim 11, further comprising a fourth muscle contraction sensor configured to measure an excitation of a fourth muscle located adjacent to the joint; and wherein the machine learning processor uses an excitation value from the fourth muscle contraction sensor in determining the set of excitation values.

13. The system of claim 1, wherein the machine learning processor is a neural network.

14. The system of claim 13, wherein the neural network is a convolutional neural network, a deep neural network, etc.

15. The system of claim 1, wherein the processor is a remote processor and wherein the system further comprises a transceiver for sending muscle contraction sensor values and/or IMU values to the remote processor.

16. The system of claim 1, wherein the machine learning processor is a part of the processor.

17. A method for determining joint dynamics of a joint of an individual, comprising:
receiving data from a first muscle contraction sensor configured to measure an excitation of a first muscle on a first side of a joint, a second muscle contraction sensor configured to measure an excitation of a second muscle located on a second side of a joint, a first movement sensor configured to measure movement on the first side of the joint, and a second movement sensor configured to measure movement on the second side of the joint; and
determining, using a machine learning processor, a set of excitation values based on an excitation value from the first muscle contraction sensor and an excitation value from the second muscle contraction sensor, wherein the set of excitation values includes an excitation value for each muscle of the joint.

18. The method of claim 17, further comprising determining, using a processor, a set of muscle-tendon unit (MTU) lengths and moment arm values from the movement sensor values.

19. The method of claim 18, further comprising determining, using a processor, a set of muscle forces based on the MTU lengths and muscle activation dynamics derived from the set of excitation values determined by the machine learning processor.

20. The method of claim 17, wherein the machine learning unit has been trained by the individual performing a set training motions.

21. The system method of claim 17, wherein the machine learning processor has been trained using movement data from a plurality of individuals.
